(19) United States
(12) Patent Application Publication
(10) Pub. No.: US 2014/0199265 A1
(43) Pub. Date: Jul. 17, 2014

(54) NUTRITIONAL COMPOSITIONS CONTAINING A NEUROLOGIC COMPONENT AND USES THEREOF

(71) Applicant: MEAD JOHNSON NUTRITION COMPANY, Glenview, IL (US)

(72) Inventors: Chenzhong Kuang, Newburgh, IN (US); Yan Xiao, Newburgh, IN (US); Eduard Poels, Newburgh, IN (US); Zeina Jouni, Evansville, IN (US); Dirk Hondmann, Evansville, IN (US)

(73) Assignee: Mead Johnson Nutrition Company, Glenview, IL (US)

(21) Appl. No.: 13/739,787

(22) Filed: Jan. 11, 2013

(51) Int. Cl. A61K 31/7072; A61K 31/164; A23L 1/29; A61K 31/7068; A61K 31/7032; A61K 31/662; A61K 31/688

(52) U.S. Cl. CPC A61K 31/7072; A61K 31/662; A61K 31/164; A61K 31/688; A61K 31/7068; A61K 31/7032; A23L 1/296
USPC 424/93.1; 514/77; 514/625; 514/49; 514/50; 514/25

(57) ABSTRACT

The present disclosure relates to nutritional compositions comprising a neurologic component, wherein, the neurologic component may promote brain and nervous system development and further provide neurological protection and repair. The neurologic component may include phosphatidylethanolamine, sphingomyelin, cytidine diphosphate-choline, ceramide, uridine, at least one ganglioside, and mixtures thereof. The disclosure further relates to methods of promoting brain and nervous system health by providing said nutritional compositions to target subjects, which includes pediatric subjects.

### TECHNICAL FIELD

The present disclosure relates to nutritional compositions that are suitable for administration to adult and pediatric subjects that include a neurologic component. The neurologic component may include phosphatidylethanolamine (“PE”), sphingomyelin, cytidine diphosphate-choline (“CDP-choline”), ceramide, uridine, at least one ganglioside, and mixtures of two or more thereof. The neurologic component provides additive and/or synergistic beneficial health benefits including enhanced brain development and improved memory, cognition, hand-eye coordination, and enhanced focusing.

Additionally, the disclosure relates to methods of promoting brain and nervous system health by providing a nutritional composition comprising the neurologic component described herein.

### BACKGROUND

The brain makes up only 2% of total body weight, yet it is a demanding organ that uses up to 30% of the day's calories and nutrients. (Harris, J. J. et al, The Energetics of CNS White Matter. Jour. of. Neuroscience, January 2012: 32(1): 356-371). The human brain and nervous system begin forming very early in prenatal life and both continue to develop until about the age of three. This early development can have lifelong effects on overall brain and nervous system health. Accordingly, brain nutrients can be important additives in the diets of infants, children and pregnant and lactating women because of their ability to promote early brain development and prevent and protect from brain and nervous system injury or illness. Additionally, brain nutrients are important for adults, as many nutrients promote nervous system repair and provide neuroprotective health benefits.

Numerous nutrients are believed to be involved with supporting healthy brain development. Recently, however, it has been discovered that PE, sphingomyelin, CDP-choline, ceramide, and uridine promote neurogenesis and/or neuronal differentiation on human adipose-derived stem cells (“hADSCs”) and human neuronal stem cells (“hNSCs”).

PE is a lipid found in biological membranes and is the second most abundant phospholipid in animal and plant tissues. It is a key building block of the membrane bilayer and is found in all living cells, although in human physiology it is found particularly in nervous tissue such as the white matter of brain, nerves, neural tissue, and in spinal cord tissues. For example, PE can amount to as much as 45% of brain phospholipids. In animal tissues, PE may exist in diacyl, alkylacyl, and alkenylacyl forms. Additionally, animal PE may contain higher portions of arachidonic (“ARA”) and docosahexaenoic acid (“DHA”) than other phospholipids, such as phosphatidylcholine.

Sphingomyelin refers to a class of sphingolipids found in animal cell membranes, particularly in the myelin sheath that surrounds nervous cell axons. In humans, sphingomyelin typically makes up 10% to 20% of plasma membrane lipids. It is believed that sphingomyelin serves to electrically insulate nerve cell axons as it makes up 25% the total lipids in the myelin sheath that surround and insulate cells of the central nervous system.

CDP-choline is a naturally occurring compound that is an essential intermediate for the synthesis of phosphatidylcholine, a major constituent of the grey matter of brain tissue. Phosphatidylcholine makes up approximately 50% of total cellular phospholipids and accounts for up to 30% of grey matter brain tissue. CDP-choline is an intermediate in the generation of phosphatidylcholine from choline. CDP-choline is biosynthesized from P-choline and cytidine triphosphate (“CTP”) by the choline-phosphate-cytidinetransferase enzyme. The formation of CDP-choline is the slowest step in the phospholipid metabolic pathway, thereby limiting the entire pathway. Thus, the cellular concentrations of CDP-choline are critical in the regulation of phospholipid biosynthesis. Cytidine and choline, which are produced from CDP-choline metabolism, are capable of crossing the blood-brain barrier and entering the central nervous system where they may be incorporated into the phospholipid fraction of neuronal cell membranes.

Ceramide refers to a family of lipid molecules composed of a sphingosine and a fatty acid. Generally, the long-chain sphingoid base is linked to a fatty acid via an amide bond. Ceramide is formed as a key intermediate in the biosynthesis of all the complex sphingolipids. More than 200 structurally distinct molecular species of ceramide have been characterized from mammalian cells. Ceramide is found in the cell membrane where it is typically concentrated preferentially into lateral liquid ordered microdomains, often referred to as “rafts” or “ceramide-rich platforms”. These ceramide rafts differ significantly in composition from other domains on the cell membrane composed of sphingomyelin or cholesterol.

Uridine is one of the four basic nucleosides found in ribonucleic acid. In the human diet, uridine is present as uridine-5-monophosphate (“UMP”). UMP is also found in the milk of mammals. Research is increasingly showing that uridine is essential for neurological growth and development. Additionally, uridine is an essential nutrient for adult brain functioning. Further, uridine is a dietary source of cytidine, a building block of both the cell membrane and phosphatidylcholine, which is necessary for memory and is a major component of cell membranes.

Gangliosides are compounds composed of glycosphingolipids with one or more sialic acid moieties, such as n-acetylneuraminic acid, linked on the sugar chain. Gangliosides consist of a hydrophobic ceramide moiety and a hydrophilic oligosaccharide chain and are components of the plasma membrane. Structurally, gangliosides concentrate on the surface of the cell membrane in structures known as “rafts”.

What is needed are nutritional compositions that comprise a neurologic component, in order to support brain and nervous system health. The neurologic component includes at least one of the following: PE, sphingomyelin, CDP-choline, uridine, ceramide, or gangliosides. These nutritional compositions may have additive and/or synergistic nervous system health benefits. Additionally, the disclosure is directed to methods of promoting and supporting brain and nervous system health by providing a nutritional composition comprising a neurologic component.

### BRIEF SUMMARY

Briefly, the present disclosure is directed, in an embodiment, to a nutritional composition comprising a neurologic component including at least one of the following: PE, sphingomyelin, CDP-choline, ceramide, uridine, and/or at least one ganglioside.

In certain embodiments the nutritional composition may further comprise DHA, lutein, zeaxanthin, resveratrol, cholesterol or mixtures of one or more thereof. It is believed that these nutrients may act synergistically with the nutrients of the neurologic component to promote neurogenesis.

Additionally, in some embodiments the nutritional composition may optionally comprise one or any combination of the following ARA, a prebiotic, a probiotic, an iron source, lactoferrin and/or β-glucan.

Due to critical brain development during the first years of life, in one embodiment the nutritional composition is an infant formula or a pediatric nutritional composition. The nutritional compositions described herein may be useful as medicaments or nutritional supplements for promoting neurological health in subjects with a neural degenerative diseases and/or brain injury. Further, the nutritional compositions of the present disclosure may provide neuroprotective health benefits and promote overall brain and nervous system health.

In some embodiments the disclosure is directed to a method for promoting brain and nervous system health, the method includes providing a nutritional composition comprising a neurologic component to the target subject.

It is to be understood that both the foregoing general description and the following detailed description present embodiments of the disclosure and are intended to provide an overview or framework for understanding the nature and character of the disclosure as it is claimed. The description serves to explain the principles and operations of the claimed subject matter. Other and further features and advantages of the present disclosure will be readily apparent to those skilled in the art upon a reading of the following disclosure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a phase contrast microscopy image of hADSCs under the neuronal differentiation condition without treatment. Morphology of hADSCs represents a condition of undifferentiation, with a large and flat morphology, as well as no obvious neurite outgrowth.

FIG. 1B is a phase contrast microscopy image of hADSCs forming morphology that resembles bi-polar, tri-polar, and multi-polar neural cells.

FIG. 2A is a phase contrast microscopy image of a control well containing hADSCs with no treatment of a neurologic component or DHA. (negative control.)

FIG. 2B is a phase contrast microscopy image of a control well containing hADSCs with treatment of DHA.

FIG. 2C is a phase contrast microscopy image of hADSCs at day 2 after treatment with PE derived from plant.

FIG. 2D is a phase contrast microscopy image of hADSCs at day 2 after treatment with PE derived from bovine.

FIG. 2E is a phase contrast microscopy image of hADSCs at day 2 after treatment with PE derived from plant in synergy with DHA.

FIG. 3A is a phase contrast microscopy image of a control well containing hADSCs with no treatment of a neurologic component.

FIG. 3B is a phase contrast microscopy image of hADSCs at day 2 after treatment with sphingomyelin derived from bovine.

FIG. 3C is a phase contrast microscopy image of hADSCs at day 2 after treatment with sphingomyelin derived from buttermilk.

FIG. 4A is a phase contrast microscopy image of a control well of hADSCs with DHA.

FIG. 4B is a phase contrast microscopy image of a control well of hADSCs with no treatment of a neurologic component or DHA.

FIG. 4C is a phase contrast microscopy image of hADSCs after treatment with CDP-choline.

FIG. 4D is a phase contrast microscopy image of hADSCs after treatment with CDP-choline and 10 μM DHA.

FIG. 4E is an inverted fluorescent microscopy image of hNSCs with no treatment.

FIG. 4F is an inverted fluorescent microscopy image of hNSCs with DHA.

FIG. 4G is an inverted fluorescent microscopy image of hNSCs with CDP-choline.

FIG. 4H is an inverted fluorescent microscopy image of hNSCs after treatment with CDP-choline and other brain nutrients, including DHA, N-octanoyl-D-threo-sphingosine, uridine, cholesterol, resveratrol, and lutein in a purified or natural form.

FIG. 5A is a phase contrast microscopy image of hADSCs after exposure to DHA.

FIG. 5B is a phase contrast microscopy image of hADSCs after exposure to N(R,S)-alpha-Hydroxydodecanoyl-D-erythrosphingosine.

FIG. 5C is a phase contrast microscopy image of hADSCs with no treatment of a neurologic component or DHA.

FIG. 5D is a phase contrast microscopy image of hADSCs after treatment with lactosylceramide.

FIG. 5E is an inverted fluorescent microscopy image of hNSCs after treatment with N-octanoyl-D-threosphingosine.

FIG. 5F is an inverted fluorescent microscopy image of hNSCs after treatment with DHA.

FIG. 5G is an inverted fluorescent microscopy image of hNSCs after treatment with N-octanoyl-D-threosphingosine and DHA.

FIG. 5H is an inverted fluorescent microscopy image of hNSCs with no treatment of a neurologic component or DHA.

FIG. 5I is an inverted fluorescent microscopy image of hNSCs after treatment with N-octanoyl-D-threosphingosine, and other brain nutrients, including DHA, CDP-choline, cholesterol, resveratrol, uridine, and lutein.

FIG. 6A is a phase contrast microscopy image of hADSCs with no treatment of neurologic component or DHA.

FIG. 6B is a phase contrast microscopy image of hADSC after treatment with uridine, observed 3 hours after switching to the neural differentiation medium with uridine.

FIG. 6C is an inverted fluorescent microscopy image of hNSCs after treatment with uridine.

FIG. 6D is an inverted fluorescent microscopy image of hNSCs after treatment with uridine, and other brain nutrients, including N-octanoyl-D-threosphingosine, DHA, CDP-choline, cholesterol, resveratrol, lutein.

### DETAILED DESCRIPTION

Reference now will be made in detail to the embodiments of the present disclosure, one or more examples of which are set forth hereinbelow. Each example is provided by way of explanation of the nutritional composition of the present disclosure and is not a limitation. In fact, it will be apparent to those skilled in the art that various modifications and variations can be made to the teachings of the present disclosure without departing from the scope of the disclosure. For instance, features illustrated or described as part of one embodiment, can be used with another embodiment to yield a still further embodiment.

Thus, it is intended that the present disclosure covers such modifications and variations as come within the scope of the appended claims and their equivalents. Other objects, features and aspects of the present disclosure are disclosed in or are apparent from the following detailed description. It is to be understood by one of ordinary skill in the art that the present discussion is a description of exemplary embodiments only and is not intended as limiting the broader aspects of the present disclosure.

The present disclosure relates generally to nutritional compositions comprising a neurologic component wherein the neurologic component may comprise PE, sphingomyelin, CDP-choline, ceramide, uridine, at least one ganglioside, or mixtures of one or more thereof. Additionally, the disclosure relates to methods of supporting and promoting brain and nervous system health, neurogenesis and neuroprotection, and cognitive development by providing a target subject a nutritional composition containing the neurologic component described herein.

“Nutritional composition” means a substance or formulation that satisfies at least a portion of a subject's nutrient requirements. The terms “nutritional(s)”, “nutritional formula (s)”, “enteral nutritional(s)”, and “nutritional supplement(s)” are used as non-limiting examples of nutritional composition(s) throughout the present disclosure. Moreover, “nutritional composition(s)” may refer to liquids, powders, gels, pastes, solids, concentrates, suspensions, or ready-to-use forms of enteral formulas, oral formulas, formulas for infants, formulas for pediatric subjects, formulas for children, growing-up milks and/or formulas for adults. The term “enteral” means deliverable through or within the gastrointestinal, or digestive, tract. “Enteral administration” includes oral feeding, intragastric feeding, transpyloric administration, or any other administration into the digestive tract. “Administration” is broader than “enteral administration” and includes parenteral administration or any other route of administration by which a substance is taken into a subject's body.

A “neurologic component” refers to a compound or compounds, or a composition, that affects neurogenesis, either by promoting or inhibiting neurogenesis. Thus, in some embodiments, a neurologic component promotes neurogenesis, while in other embodiments, a neurologic component inhibits or reduces neurogenesis.

“Pediatric subject” means a human less than 13 years of age. In some embodiments, a pediatric subject refers to a human subject that is between birth and 8 years old. In other embodiments, a pediatric subject refers to a human subject between 1 and 6 years of age. In still further embodiments, a pediatric subject refers to a human subject between 6 and 12 years of age. The term “pediatric subject” may refer to infants (preterm or fullterm) and/or children, as described below.

“Infant” means a human subject ranging in age from birth to not more than one year and includes infants from 0 to 12 months corrected age. The phrase “corrected age” means an infant's chronological age minus the amount of time that the infant was born premature. Therefore, the corrected age is the age of the infant if it had been carried to full term. The term infant includes low birth weight infants, very low birth weight infants, and preterm infants. “Preterm” means an infant born before the end of the 37^{th} week of gestation. “Full term” means an infant born after the end of the 37^{th} week of gestation.

“Child” means a subject ranging in age from 12 months to about 13 years. In some embodiments, a child is a subject between the ages of 1 and 12 years old. In other embodiments, the terms “children” or “child” refer to subjects that are between one and about six years old, or between about seven and about 12 years old. In other embodiments, the terms “children” or “child” refer to any range of ages between 12 months and about 13 years.

“Children's nutritional product” refers to a composition that satisfies at least a portion of the nutrient requirements of a child. A growing-up milk is an example of a children's nutritional product.

“Infant formula” means a composition that satisfies at least a portion of the nutrient requirements of an infant. In the United States, the content of an infant formula is dictated by the federal regulations set forth at 21 C.F.R. Sections 100, 106, and 107. These regulations define macronutrient, vitamin, mineral, and other ingredient levels in an effort to simulate the nutritional and other properties of human breast milk.

The term “growing-up milk” refers to a broad category of nutritional compositions intended to be used as a part of a diverse diet in order to support the normal growth and development of a child between the ages of about 1 and about 6 years of age.

“Nutritionally complete” means a composition that may be used as the sole source of nutrition, which would supply essentially all of the required daily amounts of vitamins, minerals, and/or trace elements in combination with proteins, carbohydrates, and lipids. Indeed, “nutritionally complete” describes a nutritional composition that provides adequate amounts of carbohydrates, lipids, essential fatty acids, proteins, essential amino acids, conditionally essential amino acids, vitamins, minerals and energy required to support normal growth and development of a subject.

Therefore, a nutritional composition that is “nutritionally complete” for a preterm infant will, by definition, provide qualitatively and quantitatively adequate amounts of carbohydrates, lipids, essential fatty acids, proteins, essential amino acids, conditionally essential amino acids, vitamins, minerals, and energy required for growth of the preterm infant.

A nutritional composition that is “nutritionally complete” for a full term infant will, by definition, provide qualitatively and quantitatively adequate amounts of all carbohydrates, lipids, essential fatty acids, proteins, essential amino acids, conditionally essential amino acids, vitamins, minerals, and energy required for growth of the full term infant.

A nutritional composition that is “nutritionally complete” for a child will, by definition, provide qualitatively and quantitatively adequate amounts of all carbohydrates, lipids, essential fatty acids, proteins, essential amino acids, conditionally essential amino acids, vitamins, minerals, and energy required for growth of a child.

As applied to nutrients, the term “essential” refers to any nutrient that cannot be synthesized by the body in amounts sufficient for normal growth and to maintain health and that, therefore, must be supplied by the diet. The term “conditionally essential” as applied to nutrients means that the nutrient must be supplied by the diet under conditions when adequate amounts of the precursor compound is unavailable to the body for endogenous synthesis to occur.

“Probiotic” means a microorganism with low or no pathogenicity that exerts at least one beneficial effect on the health of the host.

The term “inactivated probiotic” means a probiotic wherein the metabolic activity or reproductive ability of the referenced probiotic organism has been reduced or destroyed. The “inactivated probiotic” does, however, still retain, at the cellular level, at least a portion its biological glycol-protein and DNA/RNA structure. As used herein, the term “inactivated” is synonymous with “non-viable”. More specifically, a non-limiting example of an inactivated probiotic is inactivated Lactobacillus rhamnosus GG (“LGG”) or “inactivated LGG”.

“Prebiotic” means a non-digestible food ingredient that beneficially affects the host by selectively stimulating the growth and/or activity of one or a limited number of bacteria in the digestive tract that can improve the health of the host.

“β-glucan” means all β-glucan, including specific types of β-glucan, such as β-1,3-glucan or β-1,3;1,6-glucan. Moreover, β-1,3;1,6-glucan is a type of β-1,3-glucan. Therefore, the term “β-1,3-glucan” includes β-1,3;1,6-glucan.

As used herein, “non-human lactoferrin” means lactoferrin which is produced by or obtained from a source other than human breast milk. In some embodiments, non-human lactoferrin is lactoferrin that has an amino acid sequence that is different than the amino acid sequence of human lactoferrin. In other embodiments, non-human lactoferrin for use in the present disclosure includes human lactoferrin produced by a genetically modified organism. The term “organism”, as used herein, refers to any contiguous living system, such as animal, plant, fungus or micro-organism.

All percentages, parts and ratios as used herein are by weight of the total formulation, unless otherwise specified.

The nutritional composition of the present disclosure may be substantially free of any optional or selected ingredients described herein, provided that the remaining nutritional composition still contains all of the required ingredients or features described herein. In this context, and unless otherwise specified, the term “substantially free” means that the selected composition may contain less than a functional amount of the optional ingredient, typically less than 0.1% by weight, and also, including zero percent by weight of such optional or selected ingredient.

All references to singular characteristics or limitations of the present disclosure shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The methods and compositions of the present disclosure, including components thereof, can comprise, consist of, or consist essentially of the essential elements and limitations of the embodiments described herein, as well as any additional or optional ingredients, components or limitations described herein or otherwise useful in nutritional compositions.

As used herein, the term “about” should be construed to refer to both of the numbers specified as the endpoint(s) of any range. Any reference to a range should be considered as providing support for any subset within that range.

The development of the brain and nervous system plays a crucial role in the overall health and well-being of an individual. Accordingly, the nutritional composition(s) of the present disclosure promotes brain and nervous system health. Indeed, providing the neurologic component described herein can promote NSPC migration and signal transduction, increase dopamine receptor densities, support prevention of memory impairment, reduce the number of apoptotic cells, decrease neuronal degeneration, increase overall brain metabolism and reduce oxidative stress. In certain embodiments, the combination of the neurologic component and DHA has additive and/or synergistic beneficial effects that support brain and nervous system development and health.

As noted above, the neurologic component may be selected from the group consisting of PE, sphingomyelin, CDP-choline, ceramide, uridine, at least one ganglioside, and mixtures of at least two or more thereof.

Examples of PE suitable for inclusion in the neurologic component include, but are not limited to, 1,2-Dierucoyl-sn-glycero-3-phosphoethanolamine, 1,2-Dilauroyl-sn-glycero-3-phosphoethanolamine, 1,2-Dimyristoyl-sn-glycero-3-phosphoethanolamine, 1,2-Dioleoyl-sn-glycero-3-phosphoethanolamine, 1,2-Dipalmitoyl-sn-glycero-3-phosphoethanolamine, 1,2-Distearoyl-sn-glycero-3-phosphoethanolamine, 1-Palmitoyl-2-oleoyl-sn-glycero-3-phosphoethanolamine, 1-arachidonoyl-2-stearoyl-sn-glycerol 3-phospho ethanolamine, N,1-Diarachidonoyl-2-stearoyl-sn-glycerol 3-phosphoethanolamine, and phosphoethanolamine containing any fatty acid at the 1 and/or 2 positions.

PE may be present, in some embodiments in an amount from about 3.7 mg/100 kcal to about 37 mg/100 kcal. In other embodiments, PE may be present from about 10 mg/100 kcal to about 30 mg/100 kcal. In still other embodiments, PE may be present from about 15 mg/100 kcal to about 25 mg/100 kcal.

Sphingomyelin can be present in the neurological component of the nutritional composition in an amount from about 0.15 mg/100 kcal to about 73 mg/100 kcal. In other embodiments, sphingomyelin is present in the nutritional composition in an amount from about 2.9 mg/100 kcal to about 29.6 mg/100 kcal. In still other embodiments, sphingomyelin is present in the nutritional composition from about 11.1 mg/100 kcal to about 18.5 mg/100 kcal.

Examples of sphingomyelin suitable for inclusion in the neurologic component of the nutritional composition include, but are not limited to ceramide phosphorylcholine and ceramide phosphorylethanolamine, N-oleoyl sphingomyelin, N-stearoyl sphingomyelin, and/or D-erythro N-palmitoyl sphingomyelin, and mixtures thereof. For example, in one embodiment the sphingomyelin included in the neurologic component may be synthetic sphingomyelin prepared according to the procedures of U.S. Pat. No. 7,687,652 to Rochlin et al., however, the present disclosure can also include other processes for production of synthetic sphingomyelin.

When CDP-choline is present in the neurologic component, it may be present from about 7 mg/100 kcal to about 295 mg/100 kcal. In other embodiments, CDP-choline may be present from about 20 mg/100 kcal to about 76 mg/100 kcal. In still other embodiments, CDP-choline may be present from about 35 mg/100 kcal to about 50 mg/100 kcal.

Synthetic CDP-choline may be incorporated into the neurologic component. For example, in some embodiments CDP-choline may be prepared according to the procedures of U.S. Pat. No. 6,387,667 to Maruyama et al., however, the present disclosure can also include other processes for the production of synthetic CDP-choline.

Ceramide may be, in some embodiments, present in the neurological component of the nutritional composition in an amount from about 2.2 mg/100 kcal to about 22 mg/100 kcal. In other embodiments, ceramide may be present in an amount from about 4.4 mg/100 kcal to about 16.3 mg/100 kcal. In another embodiment ceramide may be present in an amount from about 7.4 mg/100 kcal to about 14.8 mg/100 kcal. In still other embodiments, ceramide may be present in an amount from about 9.6 mg/100 kcal to about 13.3 mg/100 kcal.

Examples of ceramide, suitable for inclusion in the neurologic component of the nutritional composition disclosed herein include N-octanoyl-D-threo-sphingosine, N—(R,S) alpha-Hydroxydodecanoyl-D-erythro-sphingosine, or lactosylceramide and combinations or mixtures thereof.

Uridine may be present in the neurological component of the nutritional composition in some embodiments, in an amount from about 0.15 mg/100 kcal to about 37 mg/100 kcal. In other embodiments, uridine is present in an amount from about 0.7 mg/100 kcal to about 11.1 mg/100 kcal. In another embodiment, uridine is present in the nutritional composition from about 2.9 mg/100 kcal to about 17.7 mg/100 kcal. In yet other embodiments, uridine is present in an amount from about 14.7 mg/100 kcal to about 22.2 mg/100 kcal. In still yet other embodiments, uridine is present in an amount from about 25.9 mg/100 kcal to about 37 mg/100 kcal.

Uridine, as used herein, includes but is not limited to uridine-5-monophosphate (“UMP”), uridine diphosphate, uridine diphosphoglucuronic acid and/or uridine 5′-triphosphate, and mixtures thereof.

The neurologic component may also comprise at least one ganglioside. The at least one ganglioside may be present in the nutritional composition in an amount from about 0.9 mg/100 kcal to about 14.8 mg/100 kcal. In other embodiments, the at least one ganglioside may be present from about 3 mg/100 kcal to about 10 mg/100 kcal. In still other embodiments, the at least one ganglioside may be present from about 5 mg/100 kcal to about 7.8 mg/100 kcal.

Additionally, the ganglioside included in the neurological component of the nutritional composition may be selected from those known in the art that would be compatible with the other components of the nutritional composition disclosed herein including, but not limited to, monosialogangliosides, disialogangliosides, trisialogangliosides, quadrasialogangliosides, pentasialogangliosides, and combinations thereof. Further ganglioside as used herein includes all ganglioside-functional equivalents, ganglioside-sources, ganglioside-metabolites and/or ganglioside-prerequisites.

Gangliosides are commonly defined by a short-hand nomenclature system in which “G” refers to a ganglioside, “M”, “D”, “T” “Q”, and “P” refer to mono-, di-, tri-, quadra- and pentasialogangliosides, respectively, and the subscript numbers 1, 2, 3, etc. refer to the order of migration of the gangliosides on thin-layer chromatography. The subscripts “a”, “b” and “c” indicate the series of conversion by glycosyltransferases and sialyltransferases into more complex gangliosides. Therefore, in some embodiments, the ganglioside may be selected from GM₃, GM₂, GM₁, GD₃, GD₂, GD₁a, GD₁b, GT₃, GT₂, GT₁, GT₁b, GQ₁b, GP₁, and combinations thereof. In other embodiments, the ganglioside may comprise GM₁, GD₁a, GD₁b, GT₁b, and GQ₁b and mixtures thereof.

In a particular embodiment, the at least one ganglioside of the neurologic component included in the nutritional composition may comprise GD₃ and GM₃. In this embodiment, GD₃ may comprise between about 20% and 40% of the total gangliosides of the nutritional composition and GM₃ may comprise about 20% and 40% of the total gangliosides of the nutritional composition. In another embodiment, GD₃ may comprise about 30% of the total gangliosides of the nutritional composition and GM₃ may comprise about 30% of the total gangliosides of the nutritional composition.

In yet another embodiment, the gangliosides comprise GM₃ and GD₃. In this embodiment, the GM₃ gangliosides may have a major fatty acid composition of 22:0, 18:0, 16:0, and 24:0. Similarly, the GD₃ gangliosides may have a major fatty acid composition of 18:0, 16:0, 19:0 and 22:0. In some embodiments, between about 30% and 60% of the fatty acids on the gangliosides present in the nutritional compositions of the present disclosure have a chain length of 20 or more carbon atoms. In other embodiments, between about 35% and 50% of the fatty acids on the gangliosides present in the nutritional compositions of the present disclosure have a chain length of 20 or more carbon atoms. In certain embodiments, the fatty acids of the gangliosides of the present disclosure are selected from the group consisting of long chain polyunsaturated fatty acids, oleic acid, fatty acids with 16 or fewer carbon atoms, and combinations thereof.

In some embodiments, the nutritional composition may comprise N-octanoyl-D-threo-sphingosine, N—(R,S) alpha-Hydroxydodecanoyl-D-erythrosphingosine and/or CDP-choline and further include at least one of the following: DHA, uridine, choline, cholesterol, resveratrol, or lutein, and mixtures thereof. It is believed that the combination of N-octanoyl-D-threo-sphingosine, N—(R,S) alpha-Hydroxydodecanoyl-D-erythrosphingosine, and/or ceramide with at least one of these nutrients may promote neurogenesis.

The nutrients included in the neurologic component of the nutritional composition may be formulated with other ingredients in the nutritional composition to provide appropriate nutrient levels for the target subject. In some embodiments, the nutritional composition comprising a neurologic component is a nutritionally complete formula that is suitable to support normal growth and also benefit brain development. In certain other embodiments, the composition and concentration of the nutrients in the neurologic component are designed to mimic levels that are healthy for early human development.

The nutrients of the neurological component included in the nutritional composition may include functional equivalents, sources, metabolites and/or prerequisites. Such nutrients of the neurological component may be naturally-occurring, synthetic, or developed through the genetic manipulation of organisms and/or plants, whether such source is now known or developed later.

The source for the nutrients of the neurologic component described herein may be milk, other dairy products, soybean, meats, eggs, cod, wheat germ, sugarcane extract, tomatoes, broccoli, brewer's yeast, organ meats, other plants and any other resources, fortified or not, from which the nutrients of the neurologic component could be obtained and used in a nutritional composition. Preferably, the source for the nutrients of the neurologic component should be food grade having been food derived or microorganism produced. Additionally, the source of the nutrients of the neurologic component could be part of a complex mixture obtained by separation and purification technology known in the art aimed at enrichment of the derivatives or precursors of the neurologic component nutrient of such mixtures.

Additionally, ceramide may be derived from any source known in the art for obtaining phosphatidylcholine. While the ceramide included in the neurologic component may be derived from plant or bovine sources, plant derived sources are preferred.

Further, some amounts of the nutrients in the neurologic component may be inherently present in known ingredients, such as natural oils, carbohydrate sources or proteins sources that are commonly used to make nutritional compositions. In some embodiments, the concentrations and ratios as described herein of the neurologic component are calculated based upon both added and inherent sources of the neurological component.

Additionally, the neurologic component may be added or incorporated into the nutritional composition by any method well known in the art. In some embodiments, the neurological component may be added to a nutritional composition to supplement the nutritional composition. For example, in one embodiment, the neurological component may be added to a commercially available infant formula. For example, Enfalac, Enfamil®, Enfamil® Premature Formula, Enfamil® with Iron, Enfamil® LIPIL®, Lactofree®, Nutramigen®, Pregestimil®, and ProSobee® (available from Mead Johnson & Company, Evansville, Ind., U.S.A.) may be supplemented with suitable levels of the neurologic component, and used in practice of the present disclosure.

In other embodiments, the neurologic component may be substituted for another nutrient source that does not contain the nutrients of the neurologic component. For example, a certain amount of a fat source that does not contain the neurological component may be substituted with another fat source that contains the nutrients of the neurological component. In still other embodiments, the source of an ingredient typically added to a nutritional composition may be altered, such that the source chosen provides both the ingredient that is commonly added to the nutritional composition and a nutrient of the neurological composition.

In some embodiments, the neurologic component may be included in prenatal dietary supplements. The neurologic component may be incorporated into prenatal dietary supplements by any method known in the art. The prenatal administration of the neurologic component may directly impact the development of the fetus and embryo. Since brain development begins early in prenatal life, the inclusion of the neurologic component in a prenatal dietary supplement may promote brain development and neurogenesis in pediatric subjects while still in utero.

Conveniently, commercially available prenatal dietary supplements and/or prenatal nutritional products may be used. For example, Expecta® Supplement (available from Mead Johnson & Company, Evansville, Ind., U.S.A.) may be supplemented with suitable levels of the neurologic component and used in practice of the present disclosure.

The prenatal dietary supplement may be administered in one or more doses daily. In some embodiments, the prenatal dietary supplement is administered in two doses daily. In a separate embodiment, the prenatal dietary supplement is administered in three daily doses. The prenatal dietary supplement may be administered to either pregnant women or women who are breastfeeding.

Any orally acceptable dosage form is contemplated by the present disclosure. Examples of such dosage forms include, but are not limited to pills, tablets, capsules, soft-gels, liquids, liquid concentrates, powders, elixirs, solutions, suspensions, emulsions, lozenges, beads, cachets, and combinations thereof. Alternatively, the prenatal dietary supplement of the invention may be added to a more complete nutritional product. In this embodiment, the nutritional product may contain protein, fat, and carbohydrate components and may be used to supplement the diet or may be used as the sole source of nutrition.

In some embodiments, the nutritional composition comprises at least one carbohydrate source. The carbohydrate source can be any used in the art, e.g., lactose, glucose, fructose, corn syrup solids, maltodextrins, sucrose, starch, rice syrup solids, and the like. The amount of the carbohydrate component in the nutritional composition typically can vary from between about 5 g/100 kcal and about 25 g/100 kcal. In some embodiments, the amount of carbohydrate is between about 6 g/100 kcal and about 22 g/100 kcal. In other embodiments, the amount of carbohydrate is between about 12 g/100 kcal and about 14 g/100 kcal. In some embodiments, corn syrup solids are preferred. Moreover, hydrolyzed, partially hydrolyzed, and/or extensively hydrolyzed carbohydrates may be desirable for inclusion in the nutritional composition due to their easy digestibility. Specifically, hydrolyzed carbohydrates are less likely to contain allergenic epitopes.

Non-limiting examples of carbohydrate materials suitable for use herein include hydrolyzed or intact, naturally or chemically modified, starches sourced from corn, tapioca, rice or potato, in waxy or non-waxy forms. Non-limiting examples of suitable carbohydrates include various hydrolyzed starches characterized as hydrolyzed cornstarch, maltodextrin, maltose, corn syrup, dextrose, corn syrup solids, glucose, and various other glucose polymers and combinations thereof. Non-limiting examples of other suitable carbohydrates include those often referred to as sucrose, lactose, fructose, high fructose corn syrup, indigestible oligosaccharides such as fructooligosaccharides and combinations thereof.

Moreover, the nutritional composition(s) of the disclosure may comprise at least one protein source. The protein source can be any used in the art, e.g., nonfat milk, whey protein, casein, soy protein, hydrolyzed protein, amino acids, and the like. Bovine milk protein sources useful in practicing the present disclosure include, but are not limited to, milk protein powders, milk protein concentrates, milk protein isolates, nonfat milk solids, nonfat milk, nonfat dry milk, whey protein, whey protein isolates, whey protein concentrates, sweet whey, acid whey, casein, acid casein, caseinate (e.g. sodium caseinate, sodium calcium caseinate, calcium caseinate), soy bean proteins, and any combinations thereof.

In a particular embodiment of the nutritional composition, the whey:casein ratio of the protein source is similar to that found in human breast milk. In an embodiment, the protein source comprises from about 40% to about 85% whey protein and from about 15% to about 60% casein.

In some embodiments, the nutritional composition comprises between about 1 g and about 7 g of a protein source per 100 kcal. In other embodiments, the nutritional composition comprises between about 3.5 g and about 4.5 g of protein per 100 kcal.

In some embodiments, the proteins of the nutritional composition are provided as intact proteins. In other embodiments, the proteins are provided as a combination of both intact proteins and hydrolyzed proteins, with a degree of hydrolysis of between about 4% and 10%. In certain other embodiments, the proteins are more hydrolyzed. In still other embodiments, the protein source comprises amino acids. In yet another embodiment, the protein source may be supplemented with glutamine-containing peptides. In another embodiment, the protein component comprises extensively hydrolyzed protein. In still another embodiment, the protein component of the nutritional composition consists essentially of extensively hydrolyzed protein in order to minimize the occurrence of food allergy.

In some embodiments, the protein component of the nutritional composition comprises either partially or extensively hydrolyzed protein, such as protein from cow's milk. The proteins may be treated with enzymes to break down some or most of the proteins that cause adverse symptoms with the goal of reducing allergic reactions, intolerance, and sensitization. Moreover, the proteins may be hydrolyzed by any method known in the art.

In some embodiments, the nutritional composition of the present disclosure is substantially free of intact proteins. In this context, the term “substantially free” means that the preferred embodiments herein comprise sufficiently low concentrations of intact protein to thus render the formula hypoallergenic. The extent to which a nutritional composition in accordance with the disclosure is substantially free of intact proteins, and therefore hypoallergenic, is determined by the August 2000 Policy Statement of the American Academy of Pediatrics in which a hypoallergenic formula is defined as one which in appropriate clinical studies demonstrates that it does not provoke reactions in 90% of infants or children with confirmed cow's milk allergy with 95% confidence when given in prospective randomized, double-blind, placebo-controlled trials.

The nutritional composition may be protein-free in some embodiments and comprise free amino acids as a protein equivalent source. In some embodiments, the amino acids may comprise, but are not limited to, histidine, isoleucine, leucine, lysine, methionine, cysteine, phenylalanine, tyrosine, threonine, tryptophan, valine, alanine, arginine, asparagine, aspartic acid, glutamic acid, glutamine, glycine, proline, serine, carnitine, taurine and mixtures thereof. In some embodiments, the amino acids may be branched chain amino acids. In certain other embodiments, small amino acid peptides may be included as the protein component of the nutritional composition. Such small amino acid peptides may be naturally occurring or synthesized. The amount of free amino acids in the nutritional composition may vary from about 1 g/100 kcal to about 5 g/100 kcal.

The nutritional composition may also comprise a fat source. Suitable fat or lipid sources for the nutritional composition of the present disclosure may be any known or used in the art, including but not limited to, animal sources, e.g., milk fat, butter, butter fat, egg yolk lipid; marine sources, such as fish oils, marine oils, single cell oils; vegetable and plant oils, such as corn oil, canola oil, sunflower oil, soybean oil, palm olein oil, coconut oil, high oleic sunflower oil, evening primrose oil, rapeseed oil, olive oil, flaxseed (linseed) oil, cottonseed oil, high oleic safflower oil, palm stearin, palm kernel oil, wheat germ oil; medium chain triglyceride oils and emulsions and esters of fatty acids; and any combinations thereof.

In one embodiment, the nutritional composition may contain one or more probiotics. Any probiotic known in the art may be acceptable in this embodiment. In a particular embodiment, the probiotic may be selected from any Lactobacillus species, Lactobacillus rhamnosus GG (ATCC number 53103), Bifidobacterium species, Bifidobacterium longum BB536 (BL999, ATCC: BAA-999), Bifidobacterium longum AH1206 (NCIMB: 41382), Bifidobacterium breve AH1205 (NCIMB: 41387), Bifidobacterium infantis 35624 (NCIMB: 41003), and Bifidobacterium animalis subsp. lactis BB-12 (DSM No. 10140) or any combination thereof.

If included in the composition, the amount of the probiotic may vary from about 1×10⁴ to about 1.5×10¹⁰ cfu of probiotics per 100 kcal, more preferably from about 1×10⁶ to about 1×10⁹ cfu of probiotics per 100 kcal. In certain other embodiments the amount of probitic may vary from about 1×10⁷ cfu/100 kcal to about 1×10⁸ cfu/100 kcal.

In an embodiment, the probiotic(s) may be viable or non-viable. As used herein, the term “viable”, refers to live microorganisms. The term “non-viable” or “non-viable probiotic” means non-living probiotic microorganisms, their cellular components and/or metabolites thereof. Such non-viable probiotics may have been heat-killed or otherwise inactivated, but they retain the ability to favorably influence the health of the host. The probiotics useful in the present disclosure may be naturally-occurring, synthetic or developed through the genetic manipulation of organisms, whether such source is now known or later developed.

The nutritional composition may also contain one or more prebiotics (also referred to as a prebiotic source) in certain embodiments. Prebiotics can stimulate the growth and/or activity of ingested probiotic microorganisms, selectively reduce pathogens found in the gut, and favorably influence the short chain fatty acid profile of the gut. Such prebiotics may be naturally-occurring, synthetic, or developed through the genetic manipulation of organisms and/or plants, whether such new source is now known or developed later. Prebiotics useful in the present disclosure may include oligosaccharides, polysaccharides, and other prebiotics that contain fructose, xylose, soya, galactose, glucose and mannose.

More specifically, prebiotics useful in the present disclosure may include polydextrose, polydextrose powder, lactulose, lactosucrose, raffinose, gluco-oligosaccharide, inulin, fructo-oligosaccharide, isomalto-oligosaccharide, soybean oligosaccharides, lactosucrose, xylo-oligosaccharide, chito-oligosaccharide, manno-oligosaccharide, aribino-oligosaccharide, siallyl-oligosaccharide, fuco-oligosaccharide, galacto-oligosaccharide, and gentio-oligosaccharides. In some embodiments, the total amount of prebiotics present in the nutritional composition may be from about 0.1 g/100 kcal to about 1 g/100 kcal. In certain embodiments, the total amount of prebiotics present in the nutritional composition may be from about 0.3 g/100 kcal to about 0.7 g/100 kcal. Moreover, the nutritional composition may comprise a prebiotic component comprising polydextrose (“PDX”) and/or galacto-oligosaccharide (“GOS”). In some embodiments, the prebiotic component comprises at least 20% GOS, PDX or a mixture thereof.

If PDX is used in the prebiotic composition, the amount of PDX in the nutritional composition may, in an embodiment, be within the range of from about 0.1 g/100 kcal to about 1 g/100 kcal. In another embodiment, the amount of polydextrose is within the range of from about 0.2 g/100 kcal to about 0.6 g/100 kcal. And in still other embodiments, the amount of PDX in the nutritional composition may be from about 0.1 mg/100 kcal to about 0.5 mg/100 kcal or about 0.3 mg/100 kcal.

If GOS is used in the prebiotic composition, the amount of GOS in the nutritional composition may, in an embodiment, be from about 0.1 g/100 kcal to about 1 g/100 kcal. In another embodiment, the amount of GOS in the nutritional composition may be from about 0.2 g/100 kcal to about 0.5 g/100 kcal. In other embodiments, the amount of GOS in the nutritional composition may be from about 0.1 mg/100 kcal to about 1.0 mg/100 kcal or from about 0.1 mg/100 kcal to about 0.5 mg/100 kcal.

In a particular embodiment of the nutritional composition, PDX is administered in combination with GOS. In this embodiment, PDX and GOS can be administered in a ratio of PDX:GOS of between about 9:1 and 1:9. In another embodiment, the ratio of PDX:GOS can be between about 5:1 and 1:5. In yet another embodiment, the ratio of PDX:GOS can be between about 1:3 and 3:1. In a particular embodiment, the ratio of PDX to GOS can be about 5:5. In another particular embodiment, the ratio of PDX to GOS can be about 8:2.

In a particular embodiment, GOS and PDX are supplemented into the nutritional composition in a total amount of at least about 0.2 mg/100 kcal or about 0.2 mg/100 kcal to about 1.5 mg/100 kcal. In some embodiments, the nutritional composition may comprise GOS and PDX in a total amount of from about 0.6 to about 0.8 mg/100 kcal.

As noted, the disclosed nutritional composition may comprise a source of β-glucan. Glucans are polysaccharides, specifically polymers of glucose, which are naturally occurring and may be found in cell walls of bacteria, yeast, fungi, and plants. Beta glucans (β-glucans) are themselves a diverse subset of glucose polymers, which are made up of chains of glucose monomers linked together via beta-type glycosidic bonds to form complex carbohydrates.

β-1,3-glucans are carbohydrate polymers purified from, for example, yeast, mushroom, bacteria, algae, or cereals. (Stone B A, Clarke A E. Chemistry and Biology of (1-3)-Beta-Glucans. London:Portland Press Ltd; 1993.) The chemical structure of β-1,3-glucan depends on the source of the β-1,3-glucan. Moreover, various physiochemical parameters, such as solubility, primary structure, molecular weight, and branching, play a role in biological activities of β-1,3-glucans. (Yadomae T., Structure and biological activities of fungal beta-1,3-glucans. Yakugaku Zasshi. 2000; 120:413-431.)

β-1,3-glucans are naturally occurring polysaccharides, with or without β-1,6-glucose side chains that are found in the cell walls of a variety of plants, yeasts, fungi and bacteria. β-1,3;1,6-glucans are those containing glucose units with (1,3) links having side chains attached at the (1,6) position(s). β-1,3;1,6 glucans are a heterogeneous group of glucose polymers that share structural commonalities, including a backbone of straight chain glucose units linked by a β-1,3 bond with β-1,6-linked glucose branches extending from this backbone. While this is the basic structure for the presently described class of β-glucans, some variations may exist. For example, certain yeast β-glucans have additional regions of β(1,3) branching extending from the β(1,6) branches, which add further complexity to their respective structures.

β-glucans derived from baker's yeast, Saccharomyces cerevisiae, are made up of chains of D-glucose molecules connected at the 1 and 3 positions, having side chains of glucose attached at the 1 and 6 positions. Yeast-derived β-glucan is an insoluble, fiber-like, complex sugar having the general structure of a linear chain of glucose units with a β-1,3 backbone interspersed with β-1,6 side chains that are generally 6-8 glucose units in length. More specifically, β-glucan derived from baker's yeast is poly-(1,6)-β-D-glucopyranosyl-(1,3)-β-D-glucopyranose.

Furthermore, β-glucans are well tolerated and do not produce or cause excess gas, abdominal distension, bloating or diarrhea in pediatric subjects. Addition of β-glucan to a nutritional composition for a pediatric subject, such as an infant formula, a growing-up milk or another children's nutritional product, will improve the subject's immune response by increasing resistance against invading pathogens and therefore maintaining or improving overall health.

In some embodiments, the amount of β-glucan in the nutritional composition is between about 3 mg/100 kcal and about 17 mg/100 kcal. In another embodiment the amount of β-glucan is between about 6 mg/100 kcal and about 17 mg/100 kcal.

The nutritional composition may comprise in some embodiments β-1,3;1,6-glucan. The β-1,3;1,6-glucan can be derived from baker's yeast. The nutritional composition may comprise whole glucan particle β-glucan, particulate β-glucan, PGG-glucan (poly-1,6-β-D-glucopyranosyl-1,3-β-D-glucopyranose) or any mixture thereof.

The nutritional composition of the present disclosure, may comprise lactoferrin. Lactoferrins are single chain polypeptides of about 80 kD containing 1-4 glycans, depending on the species. The 3-D structures of lactoferrin of different species are very similar, but not identical. Each lactoferrin comprises two homologous lobes, called the N- and C-lobes, referring to the N-terminal and C-terminal part of the molecule, respectively. Each lobe further consists of two sub-lobes or domains, which form a cleft where the ferric ion (Fe3+) is tightly bound in synergistic cooperation with a (bi)carbonate anion. These domains are called N1, N2, C1 and C2, respectively. The N-terminus of lactoferrin has strong cationic peptide regions that are responsible for a number of important binding characteristics. Lactoferrin has a very high isoelectric point (˜pI 9) and its cationic nature plays a major role in its ability to defend against bacterial, viral, and fungal pathogens. There are several clusters of cationic amino acids residues within the N-terminal region of lactoferrin mediating the biological activities of lactoferrin against a wide range of microorganisms.

Lactoferrin for use in the present disclosure may be, for example, isolated from the milk of a non-human animal or produced by a genetically modified organism. The oral electrolyte solutions described herein can, in some embodiments comprise non-human lactoferrin, non-human lactoferrin produced by a genetically modified organism and/or human lactoferrin produced by a genetically modified organism.

Suitable non-human lactoferrins for use in the present disclosure include, but are not limited to, those having at least 48% homology with the amino acid sequence of human lactoferrin. For instance, bovine lactoferrin (“bLF”) has an amino acid composition which has about 70% sequence homology to that of human lactoferrin. In some embodiments, the non-human lactoferrin has at least 65% homology with human lactoferrin and in some embodiments, at least 75% homology. Non-human lactoferrins acceptable for use in the present disclosure include, without limitation, bLF, porcine lactoferrin, equine lactoferrin, buffalo lactoferrin, goat lactoferrin, murine lactoferrin and camel lactoferrin.

In some embodiments, the nutritional composition of the present disclosure comprises non-human lactoferrin, for example bLF. bLF is a glycoprotein that belongs to the iron transporter or transferring family. It is isolated from bovine milk, wherein it is found as a component of whey. There are known differences between the amino acid sequence, glycosylation patters and iron-binding capacity in human lactoferrin and bLF. Additionally, there are multiple and sequential processing steps involved in the isolation of bLF from cow's milk that affect the physiochemical properties of the resulting bLF preparation. Human lactoferrin and bLF are also reported to have differences in their abilities to bind the lactoferrin receptor found in the human intestine.

Though not wishing to be bound by this or any other theory, it is believe that bLF that has been isolated from whole milk has less lipopolysaccharide (LPS) initially bound than does bLF that has been isolated from milk powder. Additionally, it is believed that bLF with a low somatic cell count has less initially-bound LPS. A bLF with less initially-bound LPS has more binding sites available on its surface. This is thought to aid bLF in binding to the appropriate location and disrupting the infection process.

bLF suitable for the present disclosure may be produced by any method known in the art. For example, in U.S. Pat. No. 4,791,193, incorporated by reference herein in its entirety, Okonogi et al. discloses a process for producing bovine lactoferrin in high purity. Generally, the process as disclosed includes three steps. Raw milk material is first contacted with a weakly acidic cationic exchanger to absorb lactoferrin followed by the second step where washing takes place to remove nonabsorbed substances. A desorbing step follows where lactoferrin is removed to produce purified bovine lactoferrin. Other methods may include steps as described in U.S. Pat. Nos. 7,368,141, 5,849,885, 5,919,913 and 5,861,491, the disclosures of which are all incorporated by reference in their entirety.

The lactoferrin that is used in certain embodiments may be any lactoferrin isolated from whole milk and/or having a low somatic cell count, wherein “low somatic cell count” refers to a somatic cell count less than 200,000 cells/mL. By way of example, suitable lactoferrin is available from Tatua Co-operative Dairy Co. Ltd., in Morrinsville, New Zealand, from FrieslandCampina Domo in Amersfoort, Netherlands or from Fonterra Co-Operative Group Limited in Auckland, New Zealand.

Surprisingly, lactoferrin included herein maintains certain bactericidal activity even if exposed to a low pH (i.e., below about 7, and even as low as about 4.6 or lower) and/or high temperatures (i.e., above about 65° C., and as high as about 120° C.), conditions which would be expected to destroy or severely limit the stability or activity of human lactoferrin. These low pH and/or high temperature conditions can be expected during certain processing regimen for nutritional compositions of the types described herein, such as pasteurization. Therefore, even after processing regimens, lactoferrin has bactericidal activity against undesirable bacterial pathogens found in the human gut.

The nutritional composition may, in some embodiments, comprise lactoferrin in an amount from about 25 mg/100 mL to about 150 mg/100 mL. In other embodiments lactoferrin is present in an amount from about 60 mg/100 mL to about 120 mg/100 mL. In still other embodiments lactoferrin is present in an amount from about 85 mg/100 mL to about 110 mg/100 mL.

The nutritional composition of the present disclosure may also contain a source of long chain polyunsaturated fatty acids (“LCPUFAs”). Suitable LCPUFAs include, but are not limited to DHA, eicosapentaenoic acid (“EPA”), ARA, linoleic (18:2 n-6), γ-linolenic (18:3 n-6), dihomo-γ-linolenic (20:3 n-6) acids in the n-6 pathway, α-linolenic (18:3 n-3), stearidonic (18:4 n-3), eicosatetraenoic (20:4 n-3), eicosapentaenoic (20:5 n-3), and docosapentaenoic (22:6 n-3).

The amount of LCPUFA in the nutritional composition is advantageously at least about 5 mg/100 kcal, and may vary from about 5 mg/100 kcal to about 100 mg/100 kcal, more preferably from about 10 mg/100 kcal to about 50 mg/100 kcal.

Sources of LCPUFAs include dairy products like eggs and butterfat; marine oils, such as cod, menhaden, sardine, tuna and many other fish; certain animal fats, lard, tallow and microbial oils such as fungal and algal oils, or from any other resource fortified or not, form which LCPUFAs could be obtained and used in a nutritional composition. The LCPUFA could be part of a complex mixture obtained by separation technology known in the art aimed at enrichment of LCPUFAs and the derivatives or precursors of LCPUFAs in such mixtures.

The LCPUFAs may be provided in the nutritional composition in the form of esters of free fatty acids; mono-, di- and tri-glycerides; phosphoglycerides, including lecithins; and/or mixtures thereof. Additionally, LCPUFA may be provided in the nutritional composition in the form of phospholipids, especially phosphatidylcholine.

In an embodiment, especially if the nutritional composition is an infant formula, the nutritional composition is supplemented with both DHA and ARA. In this embodiment, the weight ratio of ARA:DHA may be between about 1:3 and about 9:1. In a particular embodiment, the weight ratio of ARA:DHA is from about 1:2 to about 4:1.

DHA is advantageously present in the nutritional composition, in some embodiments, from at least about 17 mg/100 kcal, and may vary from about 5 mg/100 kcal to about 75 mg/100 kcal. In some embodiments, DHA is present from about 10 mg/100 kcal to about 50 mg/100 kcal.

The nutritional composition may be supplemented with oils containing DHA and/or ARA using standard techniques known in the art. For example, DHA and ARA may be added to the composition by replacing an equivalent amount of an oil, such as high oleic sunflower oil, normally present in the composition. As another example, the oils containing DHA and ARA may be added to the composition by replacing an equivalent amount of the rest of the overall fat blend normally present in the composition without DHA and ARA.

If utilized, the source of DHA and/or ARA may be any source known in the art such as marine oil, fish oil, single cell oil, egg yolk lipid, and brain lipid. In some embodiments, the DHA and ARA are sourced from single cell Martek oils, DHASCO® and ARASCO®, or variations thereof. The DHA and ARA can be in natural form, provided that the remainder of the LCPUFA source does not result in any substantial deleterious effect on the infant. Alternatively, the DHA and ARA can be used in refined form.

In an embodiment, sources of DHA and ARA are single cell oils as taught in U.S. Pat. Nos. 5,374,567; 5,550,156; and 5,397,591, the disclosures of which are incorporated herein in their entirety by reference. However, the present disclosure is not limited to only such oils.

Furthermore, some embodiments of the nutritional composition may mimic certain characteristics of human breast milk. However, to fulfill the specific nutrient requirements of some subjects, the nutritional composition may comprise a higher amount of some nutritional components than does human milk. For example, the nutritional composition may comprise a greater amount of DHA than does human breast milk. The enhanced level of DHA of the nutritional composition may compensate for an existing nutritional DHA deficit.

The disclosed nutritional composition described herein, can, in some embodiments also comprise an effective amount of iron. The iron may comprise encapsulated iron forms, such as encapsulated ferrous fumarate or encapsulated ferrous sulfate or less reactive iron forms, such as ferric pyrophosphate or ferric orthophosphate.

In some embodiments the nutritional composition(s) disclosed herein further comprises lutein. The lutein as used herein, unless otherwise specified, refers to one or more of free lutein, lutein esters, lutein salts, or other lutein derivatives of related structures as described or otherwise suggested herein. In some embodiments lutein is present from about 0.343 mg/100 kcal to about 6.0 mg/100 kcal. In still other embodiments, lutein is present from about 1.0 mg/100 kcal to about 4.0 mg/100 kcal.

Lutein sources for the present disclosure include, but are not limited to, plant sources rich in carotenoids including, but not limited to kiwi, grapes, citrus, tomatoes, watermelons, papayas and other red fruits, or dark greens, such as kale, spinach, turnip greens, collard greens, romaine lettuce, broccoli, zucchini, garden peas and brussels sprouts, spinach, and carrots. Further, sources for lutein include other plants and any other resources, fortified or not, from which lutein could be obtained and used in a nutritional composition. The lutein could be part of a complex mixture obtained by separation technology known in the art aimed at enrichment of the lutein and the derivatives or precursors of lutein in such mixtures.

Lutein for use herein includes any natural or synthetic source that is known for or is otherwise an acceptable source for use in oral nutritionals, including infant formulas. Lutein sources can be provided as individual ingredients or in any combination with other materials or sources, including sources such as multivitamin premixes, mixed carotenoid premixes, pure lutein sources, and inherent lutein components in the infant formula. The lutein concentrations and ratios as described herein may be calculated based upon both added and inherent lutein sources. In one embodiment, the nutritional composition is an infant formula which comprises at least about 10%, 25%, more preferable from about 50% to about 95%, by weight of total lutein as inherent lutein. In other embodiments, the nutritional composition is an infant formula which preferably comprises at least about 85% lutein by weight of total lutein as inherent lutein.

In certain embodiments, the nutritional composition may comprise zeaxanthin. In come embodiments zeaxanthin may be present in an amount from about 0.143 mg/100 kcal to about 4.0 mg/100 kcal. In other embodiments, zeaxanthin may be present from about 0.50 mg/100 kcal to about 3.0 mg/100 kcal. In still other embodiments zeaxanthin may be present from about 1.5 mg/100 kcal to about 2.5 mg/100 kcal. Zeaxanthin suitable for inclusion in the nutritional composition includes, but is not limited to meso-zeaxanthin (3R,3′S), and other stereoisomers such as (3R,3R′) and (3S,3′S). In some embodiments the nutritional composition may comprise lutein and zeaxanthin. The ratio of lutein to zeaxanthin may range from 95:5 to 5:95.

Cholesterol may also be present in the nutritional composition(s) of the present disclosure. In some embodiments, cholesterol is present from about 1 mg/100 kcal to about 100 mg/100 kcal. In other embodiments, cholesterol is present in the nutritional composition from about 5 mg/100 kcal to about 25 mg/100 kcal. In other embodiments cholesterol is present from about 15 mg/100 kcal to about 40 mg/100 kcal. In still other embodiments, cholesterol is present in the nutritional composition from about 50 mg/100 kcal to about 75 mg/100 kcal.

In one embodiment, cholesterol sources for the present disclosure include, but are not limited to, milk, other dairy products, eggs, meat, beef tallow, poultry, fish, shellfish and any other resources, fortified or not, from which cholesterol could be obtained and used in a nutritional composition. Sources of cholesterol also include precursors such as squalene, lanosterol, dimethylsterol, methostenol, lathosterol, and desmosterol. The cholesterol could be part of a complex mixture obtained by separation technology known in the art aimed at enrichment of the cholesterol and the derivatives or precursors of cholesterol in such mixtures.

In some embodiments, the nutritional composition of the present disclosure comprises resveratrol. Resveratrol may be present from about 5 mg/100 kcal to about 120 mg/100 kcal. In other embodiments, resveratrol may be present from about 9 mg/100 kcal to about 60 mg/100 kcal.

Resveratrol sources for the present disclosure include, but are not limited to, plant derived extracts, including but not limited to apple extract and grape seed extract. Additionally, non-limiting examples of plants rich in resveratrol suitable for use in the nutritional composition of the present disclosure include: berries (acai, grape, bilberry, blueberry, lingonberry, black currant, chokeberry, blackberry, raspberry, cherry, red currant, cranberry, crowberry, cloudberry, whortleberry, rowanberry), purple corn, purple potato, purple carrot, red sweet potato, red cabbage, eggplant. The resveratrol could be part of a complex mixture obtained by separation technology known in the art aimed at enrichment of the resveratrol and the derivatives or precursors of resveratrol in such mixtures.

Without being bound by any particular theory, it is believed that DHA, lutein, resveratrol and/or cholesterol in combination with the neurologic component may have additive and/or synergistic brain and nervous system health benefits. In certain embodiments, the nutritional composition comprising DHA, lutein, cholesterol, milk fats and/or resveratrol and mixtures thereof can act synergistically with the nutrients of the neurologic component to promote neurogenesis in nervous cell tissues.

The disclosed nutritional composition(s) may be provided in any form known in the art, such as a powder, a gel, a suspension, a paste, a solid, a liquid, a liquid concentrate, a reconstituteable powdered milk substitute or a ready-to-use product. The nutritional composition may, in certain embodiments, comprise a nutritional supplement, children's nutritional product, infant formula, human milk fortifier, growing-up milk or any other nutritional composition designed for an infant or a pediatric subject. Nutritional compositions of the present disclosure include, for example, orally-ingestible, health-promoting substances including, for example, foods, beverages, tablets, capsules and powders. Moreover, the nutritional composition of the present disclosure may be standardized to a specific caloric content, it may be provided as a ready-to-use product, or it may be provided in a concentrated form. In some embodiments, the nutritional composition is in powder form with a particle size in the range of 5 μm to 1500 μm, more preferably in the range of 10 μm to 300 μm.

If the nutritional composition is in the form of a ready-to-use product, the osmolality of the nutritional composition may be between about 100 and about 1100 mOsm/kg water, more typically about 200 to about 700 mOsm/kg water.

In certain embodiments, the nutritional composition is hypoallergenic. In other embodiments, the nutritional composition is kosher and/or halal. In still further embodiments, the nutritional composition contains non-genetically modified ingredients. In an embodiment, the nutritional formulation is sucrose-free. The nutritional composition may also be lactose-free. In other embodiments, the nutritional composition does not contain any medium-chain triglyceride oil. In some embodiments, no carrageenan is present in the composition. In other embodiments, the nutritional composition is free of all gums.

The nutritional composition of the present disclosure is not limited to compositions comprising nutrients specifically listed herein. Any nutrients may be delivered as part of the composition for the purpose of meeting nutritional needs and/or in order to optimize the nutritional status in a subject.

Moreover, in some embodiments, the nutritional composition is nutritionally complete, containing suitable types and amounts of lipids, carbohydrates, proteins, vitamins and minerals to be a subject's sole source of nutrition. Indeed, the nutritional composition may optionally include any number of proteins, peptides, amino acids, fatty acids, probiotics and/or their metabolic by-products, prebiotics, carbohydrates and any other nutrient or other compound that may provide many nutritional and physiological benefits to a subject. Further, the nutritional composition of the present disclosure may comprise flavors, flavor enhancers, sweeteners, pigments, vitamins, minerals, therapeutic ingredients, functional food ingredients, food ingredients, processing ingredients or combinations thereof.

The nutritional composition of the present disclosure may be standardized to a specific caloric content, it may be provided as a ready-to-use product, or it may be provided in a concentrated form.

In some embodiments, the nutritional composition of the present disclosure is a growing-up milk. Growing-up milks are fortified milk-based beverages intended for children over 1 year of age (typically from 1-3 years of age, from 4-6 years of age or from 1-6 years of age). They are not medical foods and are not intended as a meal replacement or a supplement to address a particular nutritional deficiency. Instead, growing-up milks are designed with the intent to serve as a complement to a diverse diet to provide additional insurance that a child achieves continual, daily intake of all essential vitamins and minerals, macronutrients plus additional functional dietary components, such as non-essential nutrients that have purported health-promoting properties.

The exact composition of a nutritional composition according to the present disclosure can vary from market-to-market, depending on local regulations and dietary intake information of the population of interest. In some embodiments, nutritional compositions according to the disclosure consist of a milk protein source, such as whole or skim milk, plus added sugar and sweeteners to achieve desired sensory properties, and added vitamins and minerals. The fat composition is typically derived from the milk raw materials. Total protein can be targeted to match that of human milk, cow milk or a lower value. Total carbohydrate is usually targeted to provide as little added sugar, such as sucrose or fructose, as possible to achieve an acceptable taste. Typically, Vitamin A, calcium and Vitamin D are added at levels to match the nutrient contribution of regional cow milk. Otherwise, in some embodiments, vitamins and minerals can be added at levels that provide approximately 20% of the dietary reference intake (DRI) or 20% of the Daily Value (DV) per serving. Moreover, nutrient values can vary between markets depending on the identified nutritional needs of the intended population, raw material contributions and regional regulations.

One or more vitamins and/or minerals may also be added in to the nutritional composition in amounts sufficient to supply the daily nutritional requirements of a subject. It is to be understood by one of ordinary skill in the art that vitamin and mineral requirements will vary, for example, based on the age of the child. For instance, an infant may have different vitamin and mineral requirements than a child between the ages of one and thirteen years. Thus, the embodiments are not intended to limit the nutritional composition to a particular age group but, rather, to provide a range of acceptable vitamin and mineral components.

In embodiments providing a nutritional composition for a child, the composition may optionally include, but is not limited to, one or more of the following vitamins or derivations thereof: vitamin B₁ (thiamin, thiamin pyrophosphate, TPP, thiamin triphosphate, TTP, thiamin hydrochloride, thiamin mononitrate), vitamin B₂ (riboflavin, flavin mononucleotide, FMN, flavin adenine dinucleotide, FAD, lactoflavin, ovoflavin), vitamin B₃ (niacin, nicotinic acid, nicotinamide, niacinamide, nicotinamide adenine dinucleotide, NAD, nicotinic acid mononucleotide, NicMN, pyridine-3-carboxylic acid), vitamin B₃-precursor tryptophan, vitamin B₆ (pyridoxine, pyridoxal, pyridoxamine, pyridoxine hydrochloride), pantothenic acid (pantothenate, panthenol), folate (folic acid, folacin, pteroylglutamic acid), vitamin B₁₂ (cobalamin, methylcobalamin, deoxyadenosylcobalamin, cyanocobalamin, hydroxycobalamin, adenosylcobalamin), biotin, vitamin C (ascorbic acid), vitamin A (retinol, retinyl acetate, retinyl palmitate, retinyl esters with other long-chain fatty acids, retinal, retinoic acid, retinol esters), vitamin D (calciferol, cholecalciferol, vitamin D₃, 1,25,-dihydroxyvitamin D), vitamin E (α-tocopherol, α-tocopherol acetate, α-tocopherol succinate, α-tocopherol nicotinate, α-tocopherol), vitamin K (vitamin K₁, phylloquinone, naphthoquinone, vitamin K₂, menaquinone-7, vitamin K₃, menaquinone-4, menadione, menaquinone-8, menaquinone-8H, menaquinone-9, menaquinone-9H, menaquinone-10, menaquinone-11, menaquinone-12, menaquinone-13), choline, inositol, β-carotene and any combinations thereof.

In embodiments providing a children's nutritional product, such as a growing-up milk, the composition may optionally include, but is not limited to, one or more of the following minerals or derivations thereof: boron, calcium, calcium acetate, calcium gluconate, calcium chloride, calcium lactate, calcium phosphate, calcium sulfate, chloride, chromium, chromium chloride, chromium picolonate, copper, copper sulfate, copper gluconate, cupric sulfate, fluoride, iron, carbonyl iron, ferric iron, ferrous fumarate, ferric orthophosphate, iron trituration, polysaccharide iron, iodide, iodine, magnesium, magnesium carbonate, magnesium hydroxide, magnesium oxide, magnesium stearate, magnesium sulfate, manganese, molybdenum, phosphorus, potassium, potassium phosphate, potassium iodide, potassium chloride, potassium acetate, selenium, sulfur, sodium, docusate sodium, sodium chloride, sodium selenate, sodium molybdate, zinc, zinc oxide, zinc sulfate and mixtures thereof. Non-limiting exemplary derivatives of mineral compounds include salts, alkaline salts, esters and chelates of any mineral compound.

The minerals can be added to growing-up milks or to other children's nutritional compositions in the form of salts such as calcium phosphate, calcium glycerol phosphate, sodium citrate, potassium chloride, potassium phosphate, magnesium phosphate, ferrous sulfate, zinc sulfate, cupric sulfate, manganese sulfate, and sodium selenite. Additional vitamins and minerals can be added as known within the art.

In an embodiment, the children's nutritional composition may contain between about 10 and about 50% of the maximum dietary recommendation for any given country, or between about 10 and about 50% of the average dietary recommendation for a group of countries, per serving, of vitamins A, C, and E, zinc, iron, iodine, selenium, and choline. In another embodiment, the children's nutritional composition may supply about 10-30% of the maximum dietary recommendation for any given country, or about 10-30% of the average dietary recommendation for a group of countries, per serving of B-vitamins. In yet another embodiment, the levels of vitamin D, calcium, magnesium, phosphorus, and potassium in the children's nutritional product may correspond with the average levels found in milk. In other embodiments, other nutrients in the children's nutritional composition may be present at about 20% of the maximum dietary recommendation for any given country, or about 20% of the average dietary recommendation for a group of countries, per serving.

The nutritional composition(s) of the present disclosure may optionally include one or more of the following flavoring agents, including, but not limited to, flavored extracts, volatile oils, cocoa or chocolate flavorings, peanut butter flavoring, cookie crumbs, vanilla or any commercially available flavoring. Examples of useful flavorings include, but are not limited to, pure anise extract, imitation banana extract, imitation cherry extract, chocolate extract, pure lemon extract, pure orange extract, pure peppermint extract, honey, imitation pineapple extract, imitation rum extract, imitation strawberry extract, grape and or grape seed extracts, apple extract, bilberry extract or vanilla extract; or volatile oils, such as balm oil, bay oil, bergamot oil, cedarwood oil, cherry oil, cinnamon oil, clove oil, or peppermint oil; peanut butter, chocolate flavoring, vanilla cookie crumb, butterscotch, toffee, and mixtures thereof. The amounts of flavoring agent can vary greatly depending upon the flavoring agent used. The type and amount of flavoring agent can be selected as is known in the art.

The nutritional compositions of the present disclosure may optionally include one or more emulsifiers that may be added for stability of the final product. Examples of suitable emulsifiers include, but are not limited to, lecithin (e.g., from egg or soy or any other plant and animal sources), alpha lactalbumin and/or mono- and di-glycerides, and mixtures thereof. Other emulsifiers are readily apparent to the skilled artisan and selection of suitable emulsifier(s) will depend, in part, upon the formulation and final product.

The nutritional compositions of the present disclosure may optionally include one or more preservatives that may also be added to extend product shelf life. Suitable preservatives include, but are not limited to, potassium sorbate, sodium sorbate, potassium benzoate, sodium benzoate, calcium disodium EDTA, and mixtures thereof.

The nutritional compositions of the present disclosure may optionally include one or more stabilizers. Suitable stabilizers for use in practicing the nutritional composition of the present disclosure include, but are not limited to, gum arabic, gum ghatti, gum karaya, gum tragacanth, agar, furcellaran, guar gum, gellan gum, locust bean gum, pectin, low methoxyl pectin, gelatin, microcrystalline cellulose, CMC (sodium carboxymethylcellulose), methylcellulose hydroxypropyl methyl cellulose, hydroxypropyl cellulose, DATEM (diacetyl tartaric acid esters of mono- and diglycerides), dextran, carrageenans, CITREM, and mixtures thereof.

The present disclosure further provides a method for promoting brain and nervous system health by providing a nutritional composition comprising a neurologic component described herein to a target subject. Without being bound by any particular theory, it is believed that providing a nutritional composition comprising the neurologic component will support neurogenesis.

In some embodiments the target subject may be a pediatric subject. Further, in one embodiment, the nutritional composition provided to the pediatric subject may be an infant formula. The neurologic component added to the infant formula may be selected from a specific source and concentrations thereof may be adjusted to maximize health benefits. In another embodiment of this method, the nutritional composition comprising a neurologic component that is provided to a pediatric subject is a growing up milk.

Studies show that total phospholipid content is significantly decreased in both the frontal cortex and hippocampus of Alzheimer's disease affected brains (20% and 10% accordingly). Additionally, researchers observed a 20% to 30% decrease of both PE and phosphatidylcholine in the frontal cortex of brains affected by Alzheimer's disease. Therefore, in one embodiment, the nutritional composition may be provided to a target subject who has been diagnosed with Alzheimer's disease or another degenerative brain disorder.

In another embodiment the nutritional composition may be provided to a target subject who has suffered, is currently suffering from, or is likely to suffer in the future from a brain and/or nervous system injury. In yet another embodiment, the nutritional composition comprising a neurologic component may be provided to any target subject to promote neuroprotection. In still other embodiments, the method is directed toward promoting neurogenesis by providing a nutritional composition comprising a neurologic component to a pregnant or lactating mother. Additionally, the nutritional compositions comprising a neurologic component described herein may provide a supplemental source of neurological nutrition to target subjects.

The methods of the present disclosure directed toward providing the nutritional compositions described herein deliver enhanced neurological nutritional and health benefits to their target subjects. The disclosure of the methods for providing the nutritional composition described herein for a particular neurological illness or to a particular target subject are not to be limiting, instead they further serve as examples where administration of the nutritional composition described herein may be appropriate.

### EXAMPLES

Examples are provided to illustrate the neurogenesis of the nutrients included in the neurologic component of the nutritional composition(s) described herein. Briefly, the neurogenesis capabilities of PE, sphingomyelin, CDP-choline, ceramide and uridine were tested on human adipose derived stem cells (“hACDSCs”) and human neuronal stem cells (“hNSCs”) by the procedure described herein. These examples should not be interpreted as any limitation on the nutritional compositions disclosed herein, but serve as illustrations of neurogenesis of the neurologic component. It is intended that the specification, together with the example, be considered to be exemplary only, with the scope and spirit of the disclosure being indicated by the claims which follow the examples. The procedures of U.S. patent application Ser. No. 13/408,485 filed by Kuang, et al. and U.S. patent application Ser. No. 13/408,490 filed by Kuang, et al. may be suitable for practice of the present disclosure and are hereby incorporated by reference.

### Example 1

This example describes the neurogenesis of hADSCs by PE as compared to DHA and a negative control.

PE from bovine and plant was purchased from Matreya® (Cat.#1069) and (Cat.#1301) respectively. PE from bovine was diluted in 100% ethanol to 67.2 mM. PE from plant was diluted in 100% ethanol to 67.6 mM. These solutions were then stored at 4°-8° C.

hADSCs were purchased from Invitrogen, also known as Life Technologies, of Carlsbad, Calif., U.S.A., and were cultured as near confluent monolayers in 100 mm culture plates within a maintenance media consisting of Complete MesenPro RS medium with growth supplement and L-glutamine obtained from Invitrogen®. The process of culturing, passage, and seeding the hADSCs is described below.

The subculture of hADSCs was performed when cell culture reached confluence. To passage hADSCs, the following procedure is used: i) aspirate the Complete MesenPRO RS medium from the cells; ii) rinse the surface area of the cell layer with Dulbecco's phosphate buffered saline (DBPS) buffer by adding the DPBS to the side of the vessel opposite the attached cell layer and rocking the vessel back and forth several times; iii) remove the DPBS by aspiration and discard; iv) detach the cells by adding a sufficient volume of pre-warmed trypsin-EDTA solution without phenol red to cover the cell layer; v) incubate at 37° C. for approximately 7 minutes; vi) observe the cells under a microscope to determine if additional incubation is needed; vii) add 3 mL of the maintenance media to the plate, mix the cell suspension, add the suspension to a 15 mL centrifuge tube and centrifuge at 210 g for 5 minutes; viii) determine the total number of cells and percent viability using a hemacytometer; ix) add Complete MesenPRO RS medium to each vessel so that the final culture volume is 0.2 mL-0.5 mL per cm²; x) seed the cells by adding the appropriate volume of cells to each vessel and incubate at 37° C., 5% CO₂ and 90% humidity; and xi) three or four days after seeding, completely remove the medium and replace with an equal volume of Complete MesenPRO RS medium.

Before seeding the passaged hADSCs on fresh culture plates, the surfaces of the culture ware are washed with sterile DPBS solution three times, followed by multiple rinses with sterile water. The first layer of coating is poly-L-ornithine. The coating is prepared by adding about 15 to about 20 μg/mL of poly-L-ornithine and incubating at 37° C. for one hour. The plate is washed three times with DPBS, 15 minutes per wash. The second layer of coating is bovine plasma fibronectin. The fibronectin is diluted in DPBS from stock to 1:1000 and 500 μL is added to each well. The plate is left at room temperature for one hour. One final wash with 500 μL per well of DPBS is performed and the plate is used immediately.

The cells were then subjected to removal and reseeded at a density of 2×10⁴ cells/ml (1×10⁴ cells/well) onto 24-well culture plates that contained a poly-L-ornithine and bovine plasma fibronectin coating.

Three days after seeding and priming; the culture medium was changed into neuronal differentiation medium. The culture plates were removed from the incubator and all procedures were conducted in a laminar flow hood. The culture medium was completely removed from each well. The hADSCs were then washed with sterile DPBS solution in an amount of about 1 ml per well, to remove excess culture medium. The DPBS solution was removed and replaced with neuronal differentiation medium. The formulation of the neuronal differentiation medium is such that neurogenesis would be attributed to the nutrient and not to the medium. The neuronal differentiation medium used was Neurobasal™ Medium, available from Invitrogen®, which comprises the following ingredients listed below in Table 1.

TABLE 1
Neurobasal ™ Medium

| | Molecular | Concentration | |
|---|---|---|---|
| Components | Weight | (mg/L) | mM |
| Amino Acids | | | |
| Glycine | 75 | 30 | 0.4 |
| L-Alanine | 89 | 2 | 0.0225 |
| L-Arginine hydrochloride | 211 | 84 | 0.398 |
| L-Asparagine-H₂O | 150 | 0.83 | 0.00553 |
| L-Cysteine | 121 | 31.5 | 0.26 |
| L-Histidine hydrochloride-H₂O | 210 | 42 | 0.2 |
| L-Isoleucine | 131 | 105 | 0.802 |
| L-Leucine | 131 | 105 | 0.802 |
| L-Lysine hydrochloride | 183 | 146 | 0.798 |
| L-Methionine | 149 | 30 | 0.201 |
| L-Phenylalanine | 165 | 66 | 0.4 |
| L-Proline | 115 | 7.76 | 0.0675 |
| L-Serine | 105 | 42 | 0.4 |
| L-Threonine | 119 | 95 | 0.798 |
| L-Tryptophan | 204 | 16 | 0.0784 |
| L-Tyrosine | 181 | 72 | 0.398 |
| L-Valine | 117 | 94 | 0.803 |
| Vitamins | | | |
| Choline chloride | 140 | 4 | 0.0286 |
| D-Calcium pantothenate | 477 | 4 | 0.00839 |
| Folic Acid | 441 | 4 | 0.00907 |
| Niacinamide | 122 | 4 | 0.0328 |
| Pyridoxine hydrochloride | 204 | 4 | 0.0196 |
| Riboflavin | 376 | 0.4 | 0.00106 |
| Thiamine hydrochloride | 337 | 4 | 0.0119 |
| Vitamin B12 | 1355 | 0.0068 | 0.000005 |
| i-Inositol | 180 | 7.2 | 0.04 |
| Inorganic Salts | | | |
| Calcium Chloride (CaCl₂) | 111 | 200 | 1.8 |
| (anhyd.) | | | |
| Ferric Nitrate (Fe(NO₃)3″9H₂O) | 404 | 0.1 | 0.000248 |
| Magnesium Chloride | 95 | 77.3 | 0.814 |
| (anhydrous) | | | |
| Potassium Chloride (KCl) | 75 | 400 | 5.33 |
| Sodium Bicarbonate (NaHCO₃) | 84 | 2200 | 26.19 |
| Sodium Chloride (NaCl) | 58 | 3000 | 51.72 |
| Sodium Phosphate monobasic | 138 | 125 | 0.906 |
| (NaH₂PO4—H₂O) | | | |
| Zinc sulfate (ZnSO₄—7H₂O) | 288 | 0.194 | 0.000674 |
| Other Components | | | |
| D-Glucose (Dextrose) | 180 | 4500 | 25 |
| HEPES | 238 | 2600 | 10.92 |
| Sodium Pyruvate | 110 | 25 | 0.227 |

PE was added to individual wells at various concentrations in the serum-free medium. Pre-warmed serum-free medium contains Neural Basal medium with L-glutamine, 20 ng/mL of bFGF, 20 ng/mL of EGF and N2 supplement. See Table 2 below.

TABLE 2
N2 Supplement.

| | Molecular | Concentration | |
|---|---|---|---|
| Components | Weight | (mg/L) | mM |
| Proteins | | | |
| Human transferrin (Holo) | 10000 | 10000 | 1 |
| Insulin recombinant full chain | 5807.7 | 500 | 0.0861 |
| Other components | | | |
| Progesterone | 314.47 | 0.63 | 0.002 |
| Putrescine | 161 | 1611 | 10.01 |
| selenite | 173 | 0.52 | 0.00301 |

Treatments of PE, from both bovine and plant, were tested at concentrations of 10 μM, 20 μM, and 40 μM. PE in varying concentrations was tested individually and compared to the positive control, DHA, and the negative control (no treatment) under phase contrast microscopy at 24 hours, 48 hours and 96 hours. The experiments were repeated in triplicate.

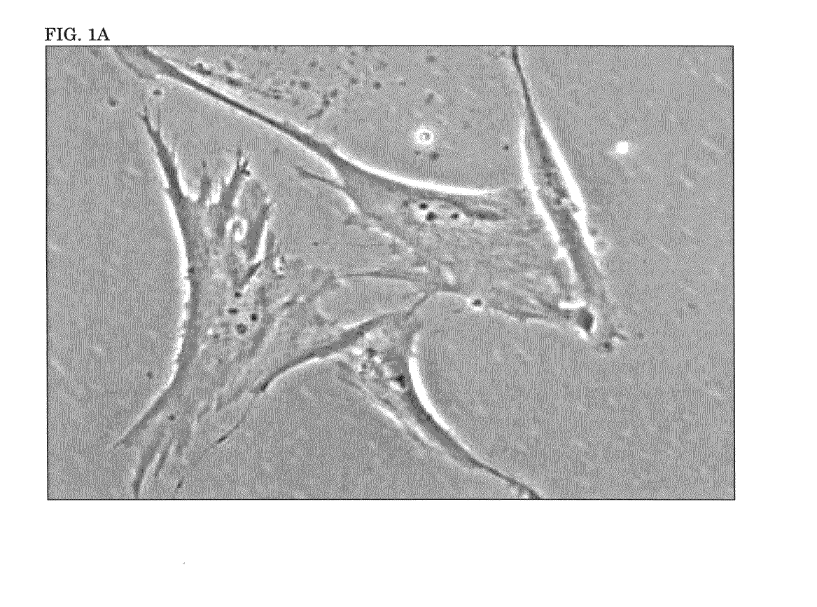

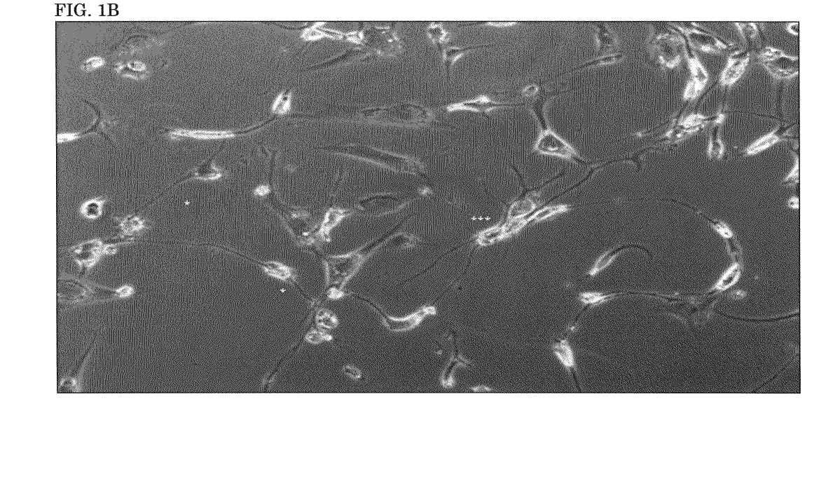

After images were collected, data analysis and comparison was made to determine the effectiveness of each PE in promoting neurogenesis. Neuronal differentiation is determined by neuronal morphology. Some of these changes include shrinkage of the cytoplasm, and formation of axons and dendrite-like cytoplasmic projections (neurites). These changes begin with the cytoplasm of hADSCs retracting towards the nucleus to form contracted cell bodies with cytoplasmic extensions. Cells eventually develop a morphology that resembles bi-polar, tri-polar and multi-polar neuronal cells. See FIGS. 1A and 1B.

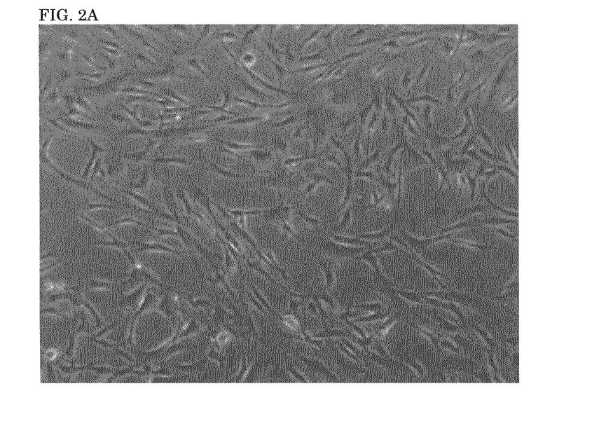

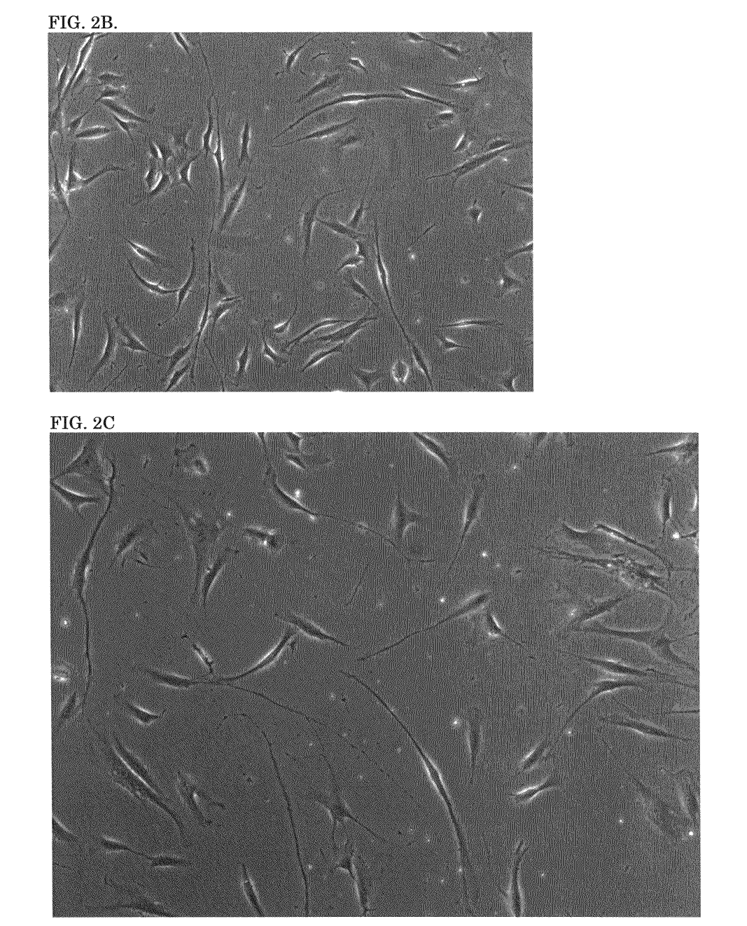

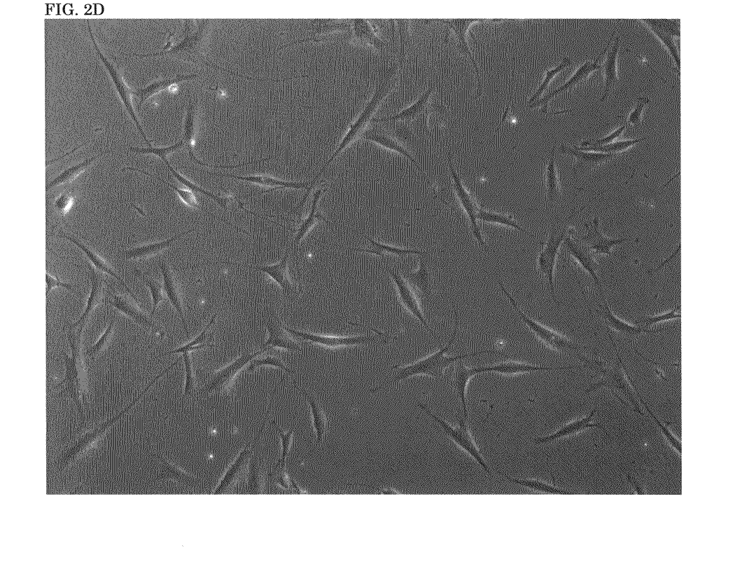

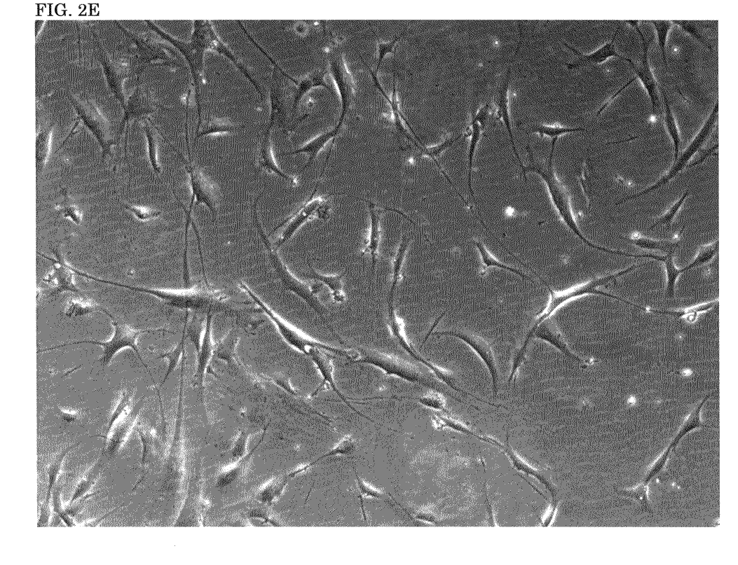

Generally, if the hADSCs display neuronal morphology this result is attributed to the neurogenesis capability of the neurologic component added, in this example PE. For example, the hADSCs in the control wells with no treatment maintained their putative morphology as large, flat and spread cells on the culture surface, suggesting no obvious neurogenesis. See. FIG. 2A.

Noticeably, among the additions of PE at various aforementioned concentrations, PE at a concentration of 20 μM demonstrated the strongest effect to enhance neurogenesis as shown by the neuronal morphology displayed by the hADSCs in FIGS. 2C and 2D. In light of these results, it was determined that PE can serve as a naturally-occurring nutrient that possesses neurogenesis actions. The addition of PE, both from plant (FIG. 2C) and bovine (FIG. 2D) also promoted neurogenesis when compared to the negative control. Among the additions of PE at various aforementioned concentrations, as illustrated, PE at a concentration of 20 μM demonstrated the strongest effect to enhance neurogenesis, showing extensive neurite outgrowth, shrinkage of cytoplasm and neuronal differentiation.

The additions of DHA at 10 μM to hADSCs as a positive control enhanced neuronal morphology of hADSCs when compared to the negative control. Further, in the presence of DHA at 10 μM, a few of the hADSCs changed dramatically from their putative morphology into neuronal cell morphology as the cytoplasm shrank and neurities began to protrude from the hADSCs. See FIG. 2B. When treated hADSCs with the combination of PE from bovine at 20 μM and DHA at 10 μM, the synergy of two nutrients enhance neurogenesis further with longer protruding neurite outgrowth and multipolar neuronal differentiation (FIG. 2E).

### Example 2

This example describes the neurogenesis of hADSCs by sphingomyelin as compared to DHA and a negative control.

Sphingomyelin from egg (Cat. #1332) and buttermilk (Cat.#1329) was purchased from Matreya (Pleasant Gap, Pa., USA). Sphingomyelin from egg and buttermilk was diluted in 100% ethanol to 13.7 mM, individually. The hADSCs were cultured, passaged, seeded and subjected to sphingomyelin via the same procedure outlined in Example 1.

hADSCs including 10 μM sphingomyelin, 20 μM sphingomyelin, 40 μM sphingomyelin, 10 μM DHA and the negative control were observed under phase contrast microscopy at 24 hours, 48 hours, and 96 hours after treatment.

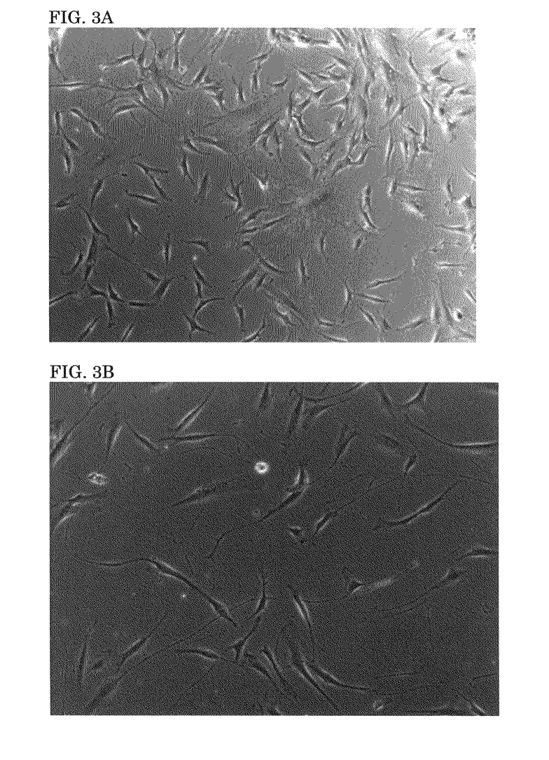

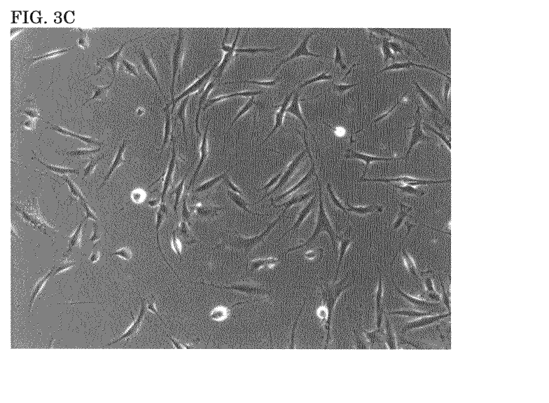

Even at low concentrations of sphingomyelin, most extensions, although not extremely long, were longer than negative control and much more numerous. Sphingomyelin from bovine at 40 μM and from buttermilk at 20 μM were more effective than DHA in this protocol. See. FIGS. 3A, 3B, and 3C.

Among the additions of sphingomyelin at various aforementioned concentrations it was found that 40 μM of sphingomyelin enhanced neural differentiation of hADSCs. In light of these results, it was determined that sphingomyelin can serve as a naturally-occurring nutrient that possesses neurogenesis actions.

### Example 3

This example describes the neurogenesis of hADSCs by CDP-choline as compared to DHA and a negative control.

CDP-choline was obtained from Kyowa Hakko Gio Co. CDP-choline was dissolved to 200 μM in sterile H₂O in a laminar flow hood, giving a clear stock solution. The hADSCs were cultured, passaged, seeded and subjected to CDP-choline via the same procedure outlined in Example 1.

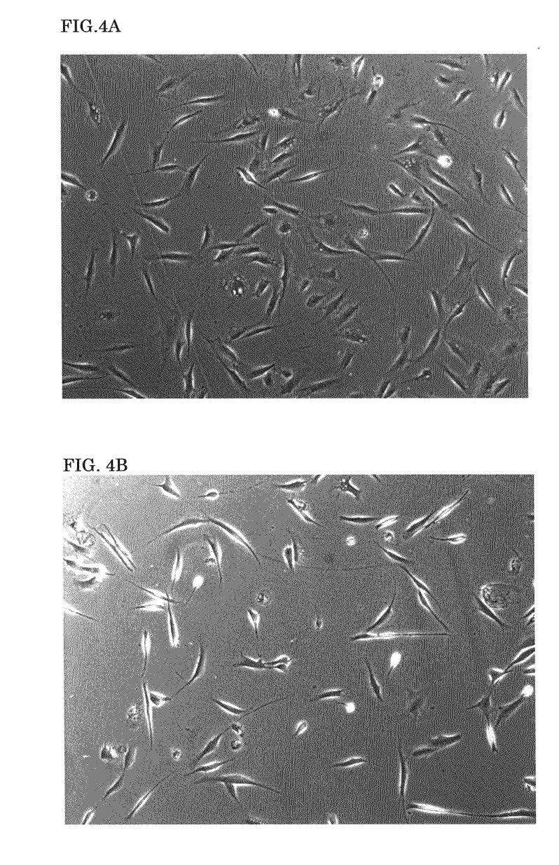

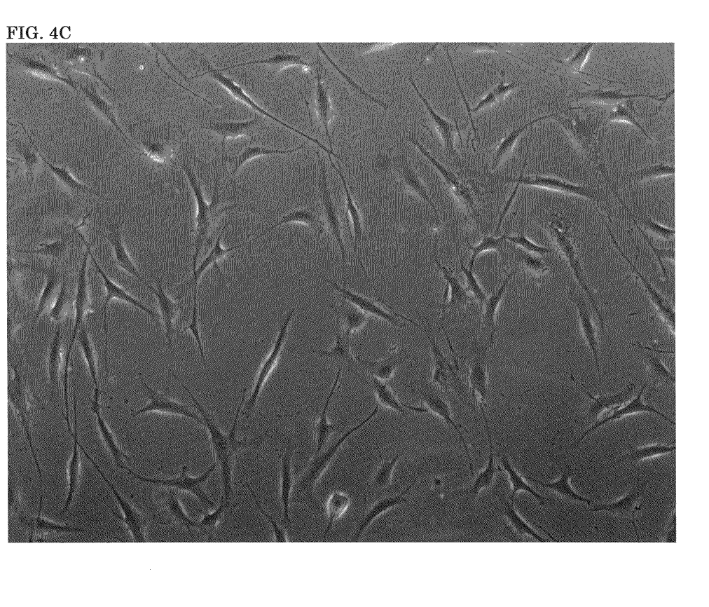

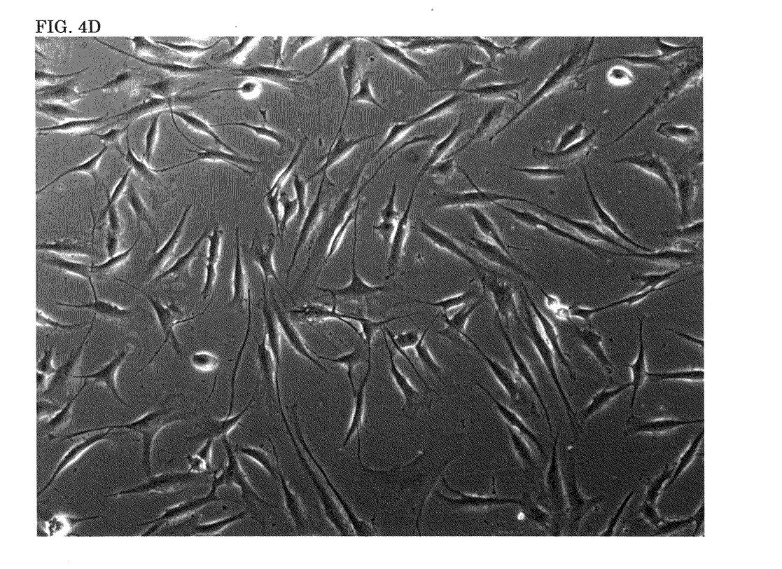

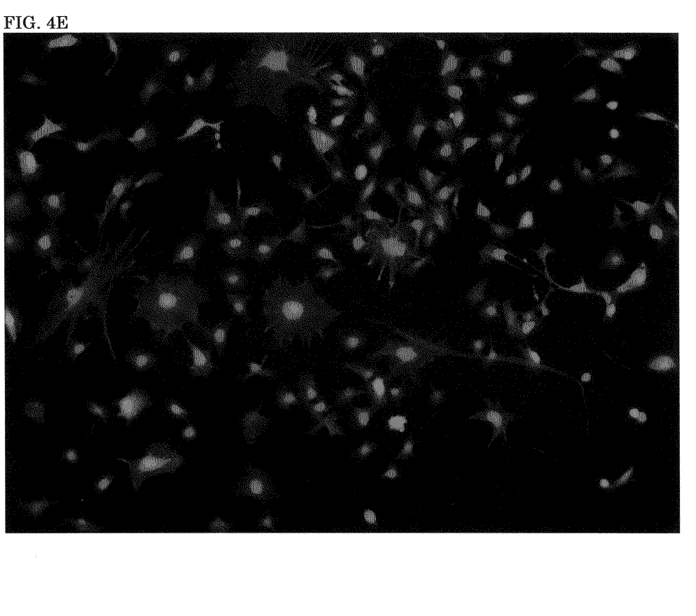

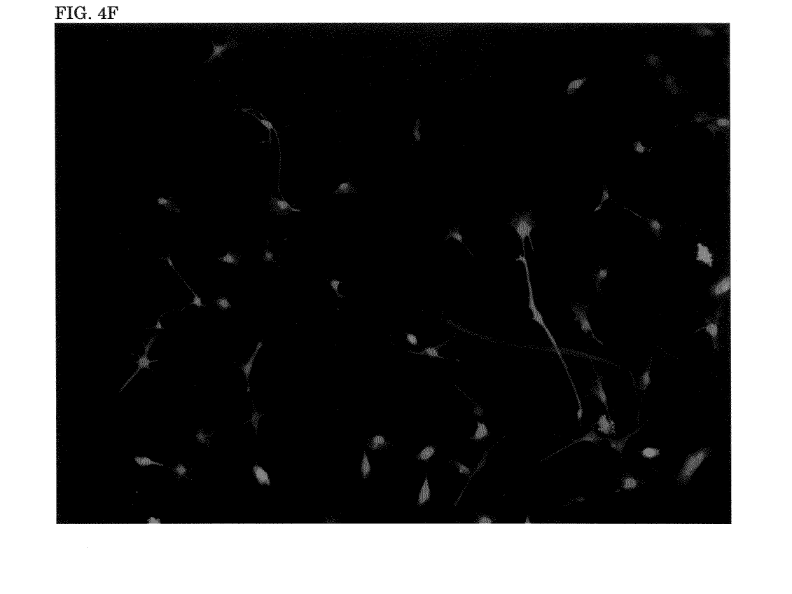

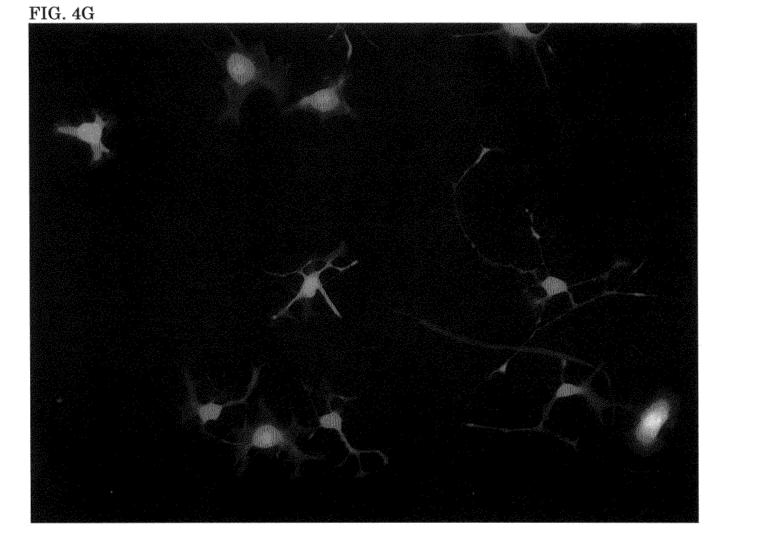

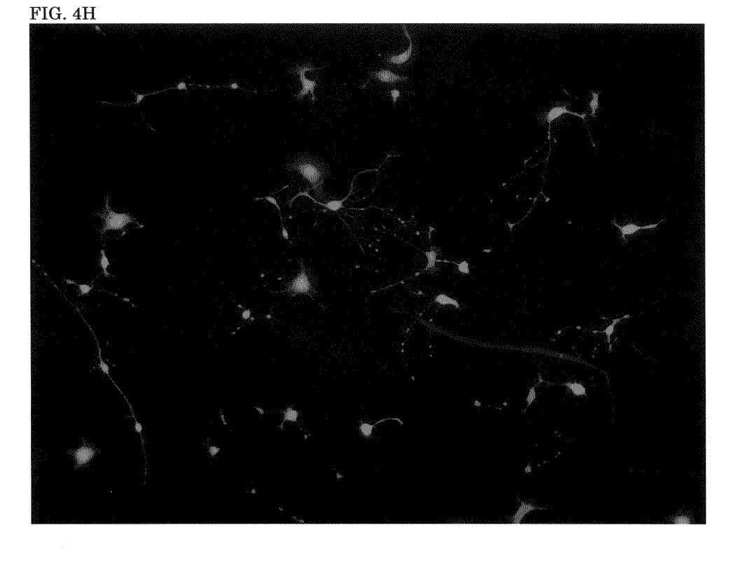

Treatments of CDP-choline, were tested at concentrations of 5 μM and 10 μM. CDP-choline in varying concentrations was tested individually and compared to the positive control, DHA at 10 μM (FIG. 4A), and the negative control (FIG. 4B) under phase contrast microscopy at 3 hours, 24 hours and 48 hours after treatment. The experiments were repeated in triplicate.

Additionally, CDP-choline at the concentration of 5 μM demonstrated an effect to enhance neurogenesis as observed neurite outgrowth and neuronal morphological changes were observed on hADCSs treated with CDP-choline. See. FIG. 4C. Note that the cytoplasm shrank and neurites began to protrude. The corolla of light can be observed with the neuronal differentiated cells due to the shrinking cellular body and the enhanced reflection of light from the microscope. Longer neurite growth was observed.

CDP-choline at 10 μM in addition with DHA at 10 μM exhibited synergistic neurogenesis in hADSCs. See. FIG. 4D. The hADSCs underwent significant neurogenesis in the presence of the combination of CDP-choline and DHA.

Additionally, CDP-choline promotes neurogenesis on human neuronal stem cells (“hNSCs”) line. Disclosed herein is the method for testing neurogenesis of CDP-choline on hNSCs and the results obtained.

Briefly, hNSCs were purchased from Millipore, Bellerica, Mass., U.S.A., with genetic modification to constitutively express green fluorescent protein (“GFP”). The hNSCs were cultured on laminin coated plates as recommended by the manufacturer. Both laminin and DEME/F12 were obtained from Millipore. Laminin was diluted with DMEM/F12 to 20 μg/mL. 10 ml of diluted laminin solution was added to 10 cm tissue culture dish. Then the culture dish was incubated in a 37° C., 5% CO2 incubator overnight. Just before use, the laminin solution was aspirated and rinsed once with sterile DPBS solution. hNSCs were cultured in the ReNcell NSC maintenance medium (Millipore) supplied with 20 ng/mL bFGF and 20 ng/mL EGF in a 37° C., 5% CO2 incubator. Medium was exchanged with fresh medium containing bFGF and EGF every other day thereafter. The cells reached 80% confluence 2 to 3 days after this step.

After hNSCs reach 80% confluence, hNSCs were ready for the differentiation experiment. Before seeding, a 96-well plate was freshly coated with 20 μg/mL laminin solution followed by a brief DPBS rinse as described above. Culture medium was removed carefully and hNSCs were dissociated within 3 ml Accutase (Millipore) in a 37° C., 5% CO2 incubator for 3 minutes. Then 5 ml of ReNcell NSC maintenance medium (Millipore) supplied with 20 ng/mL bFGF and 20 ng/mL EGF were added. The cell suspension was then transferred a sterile 15 ml conical tube and the cells were pelleted by the centrifugation at 300×g for 5 minutes. Supernatant was removed. 2 ml medium was then applied to the tube and hNSCs was resuspended thoroughly. The hNSCs were seeded on a 96-well plate at a density of 1×10⁴ cell/ml (1000 cells/well, 100 μl/well).

After attaching to the culture surface, the culture medium was switched to serum-free differentiation medium in the presence of CDP-choline, or DHA, or no treatment. The serum-free differentiation medium was prepared freshly before the switching including 40 ml DMEM/F12 (Millipore), 400 μL-Glutamine at a concentration of 200 mM (Life Technologies, Carlsbad, Calif.), 400 μl B27 solution (Life Technologies, Carlsbad, Calif.), and 40 μl Heperin (Sigma-Aldrich, St. Louis, Mich.) solution at a concentration of 10 mg/mL.

The cells were observed for morphological changes after 72 hours under inverted fluorescent microscopy. The entire 96-well plate was placed under the Leica DMI4000B fluorescent microscopy, and images were taken with GFP filter under a microscope with UV light source.

CDP-choline of 404 dramatically promoted neurogenesis when compared to no treatment. See FIG. 4E. The hNSCs observed had shrinking cellular bodies, projecting neurities and were developing dendrites. See FIG. 4F. The length of neurite outgrowth in the presence of CDP-choline is comparable to DHA at 20 μM which demonstrates good effects on neurogenesis. See FIG. 4G.

Further, CDP-choline at 12.5 μM synergizes with other brain nutrients including DHA at 5 μM, N-octanoyl-D-threo-sphingosine at 0.1 μM, uridine at 20 μM, cholesterol at 25 μM, resveratrol at 8.8 μM, and lutein at 0.3 μM to dramatically promote neurogenesis in a rapid manner. The morphological changes shown in FIG. 4H, illustrates the neuronal morphological changes of hNSCs after treatment with CDP-choline and these other brain nutrients. These morphological changes include the appearance of more oligodendrocyte differentiation, which suggest myelination function.

### Example 4

This example describes the neurogenesis of hADSCs by ceramide as compared to DHA and a negative control. The ceramides used for the following experiments were N—(R,S)-alpha-Hydroxydodecanoyl-D-erythrosphingosine, lactosylceramide and N-octanoyl-D-threo-sphingosine.

Disclosed herein is the method for testing neurogenesis of N—(R,S)-alpha-Hydroxydodecanoyl-D-erythrosphingosine on hADSCs and the results obtained.

N—(R,S)-alpha-Hydroxydodecanoyl-D-erythrosphingosine was purchased from Matreya® (Cat. #2042) and dissolved in 100% ethanol at 1 mM and stored as stock solution at −80° C. to avoid changes in physical and chemical nature. The same procedure as outlined in Example 1 was used to culture, passage, seed the cells and subject them to N—(R,S)-alpha-Hydroxydodecanoyl-D-erythrosphingosine, except that the cells were seeded at a density of 2×10⁴ cells/ml (1×10⁴ cells/well) onto 24-well culture plates that contained a poly-L-ornithine and bovine plasma fibronectin coating.

Treatments of N—(R,S)-alpha-Hydroxydodecanoyl-D-erythrosphingosine, were tested at concentrations of 20 μM and 40 μM and compared to the positive control, DHA at 10 μM, and the negative control under phase contrast microscopy at 24 hours after treatment. The experiments were repeated in triplicate.

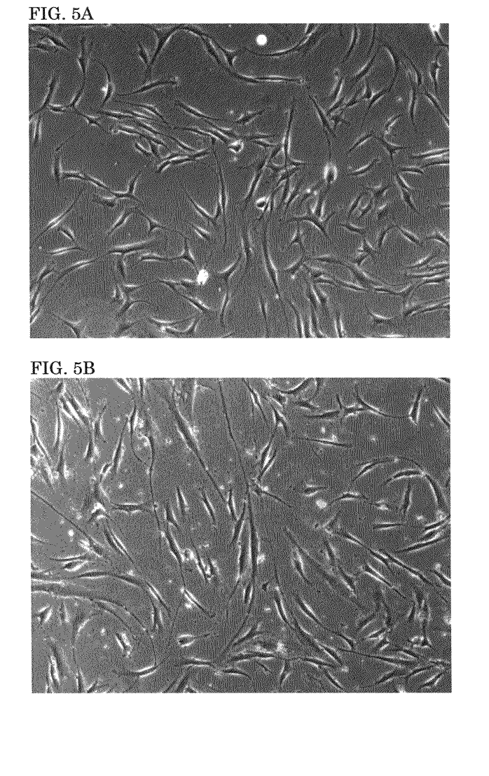

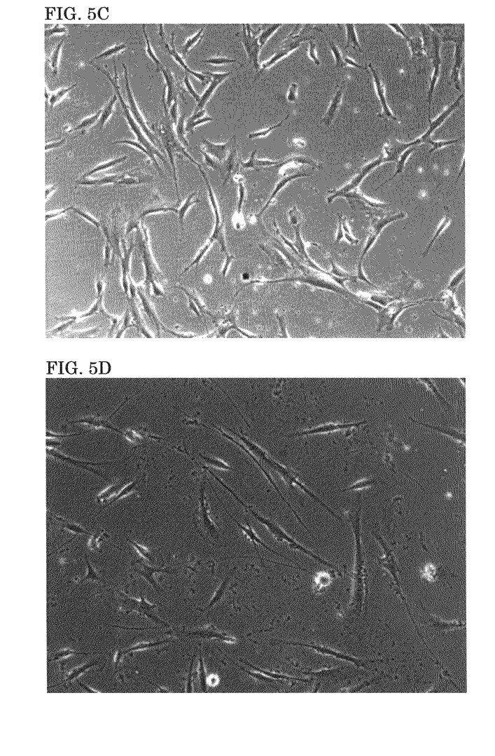

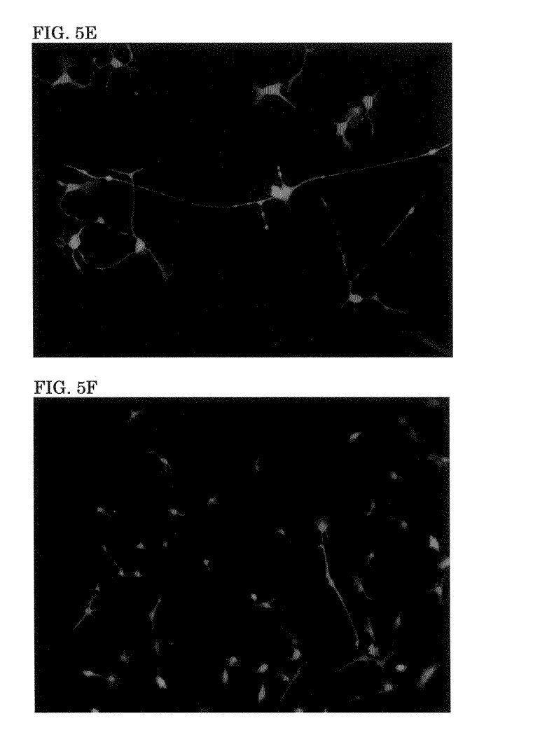

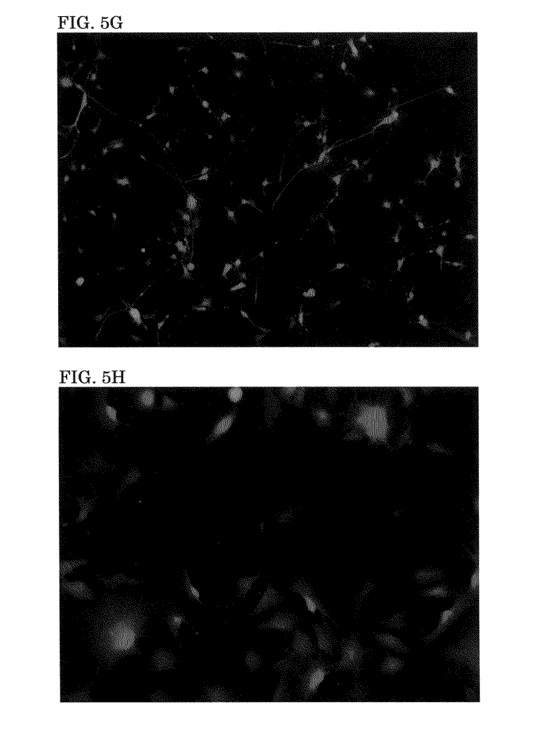

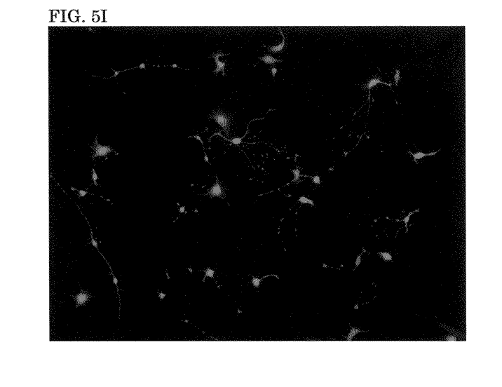

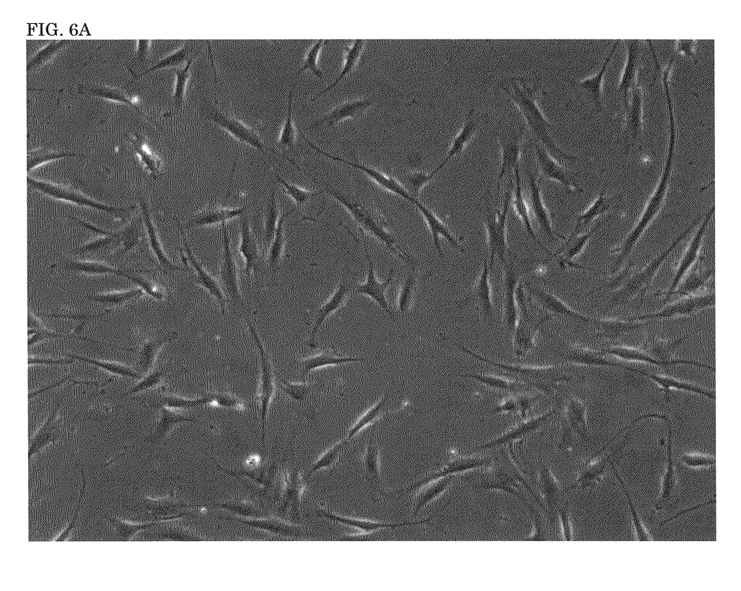

Early neurogenesis was first observed 24 hours after switching to the neural differentiation medium with the treatments of DHA at a concentration of 10 μM. See FIG. 5A.

Early neurogenesis was also, observed 24 hours after treating the hADSCs with N—(R,S)-alpha-Hydroxydodecanoyl-D-erythrosphingosine at a concentration of 40 μM See FIG. 5B.

The hADSCs, in the control wells with no treatment maintained their putative morpohology as large, flat and spread cells on the culture surface, suggesting no obvious neurogenesis at this time point. See FIG. 5C.

Further, the treatment of N—(R,S)-alpha-Hydroxydodecanoyl-D-erythrosphingosine, when compared with DHA, showed moderate effects to promote neurogenesis. Additionally, the treatment of hADSCs with N—(R,S)-alpha-Hydroxydodecanoyl-D-erythrosphingosine did show obvious neuronal changes in terms of morphology, suggesting that N—(R,S)-alpha-Hydroxydodecanoyl-D-erythrosphingosine may have a different neurogenesis pathway from that of DHA.

Similarly, lactosylceramide was found to promote neurogenesis on hADSCs. Lactosylceramide, also known as lactocerebroside, is found in small amounts in most animal tissues, but has a number of significant biological functions, and is of great importance as the biosynthetic precursor of most of the neural oligoglycosylceramides, sulfatides and gangliosides.

Lactosylceramide was obtained from Metreya (Cat. #1500) and dissolved in 100% ethanol at a concentration of 9 mM. Using the aforementioned procedure as described above for N—(R,S)-alpha-Hydroxydodecanoyl-D-erythrosphingosine, lactosylceramide at a testing concentration of 10 μM was found to exhibit neurogenesis action on hADSCs. See. FIG. 5D.

Additionally, N-octanoyl-D-threo-sphingosine was found to promote neurogenesis on hNSCs. N-octanoyl-D-threo-sphingosine was dissolved in 100% ethanol at 11.7 mM and stored at −80° C. as a stock solution. Human neuronal stem cells were purchased from Millipore® with a genetic modification to constitutively express green fluorescent protein. The hNSCs were cultured, seeded and exposed to N-octanoyl-D-threo-sphingosine according to the procedure described in Example 3.

N-octanoyl-D-threo-sphingosine at 10 μM dramatically promoted neurogenesis as the hNSCs appeared with shrinking cellular bodies, projecting neurites and developing dendrites. See FIG. 5E.

Moreover, N-octanoyl-D-threo-sphingosine at an experimental concentration of 10 μM is synergetic with 10 μM DHA to further promote neurogenesis. See. FIG. 5G. This is compared to the positive control group containing DHA alone See FIG. 5F, and the negative control group containing no treatment. See FIG. 5H.

Additionally, it was discovered that N-octanoyl-D-threo-sphingosine is able to synergize with other brain nutrients to dramatically promote neurogenesis as early as three hours after application. See. FIG. 5I, which shows the neuronal morphological changes 3 hours after application of 0.1 μM N-octanoyl-D-threo-sphingosine together with 12.5 μM CDP-choline and other brain nutrients, including 5 μM DHA, 20 μM uridine, 25 μM cholesterol, 8.8 μM resveratrol, and 0.3 μM lutein in a purified or natural form. The neuronal morphological changes are shown as appearing more toward oligodendrocyte differentiation, suggesting a myelination function.

### Example 5

This example describes the neurogenesis of hADSCs by uridine as compared to DHA and a negative control. This example also described the neurogenesis of hNSCs by uridine.

Uridine was purchased from Sigma-Aldrich® (Cat. #U3003) and dissolved in sterile water to 50 mg/ml in a laminar flow hood, giving a clear stock solution. The hADSCs were cultured, passaged, seeded and exposed to uridine according to the procedure described in Example 1.

Uridine at 20 μM was tested individually and compared to the positive control, DHA at 20 μM, and no treatment as the negative control. Uridine and DHA were added directly to the neural differentiation medium three days after the hADSCs were seeded onto the coated culture surfaces. The samples were observed under phase contrast microscopy at 3 hours, 24, hours and 48 hours after the treatment. The experiments were repeated in triplicate.

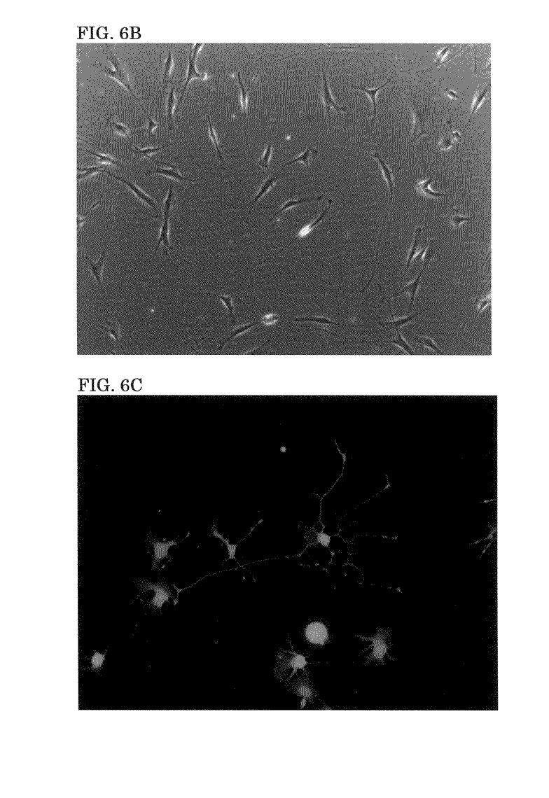

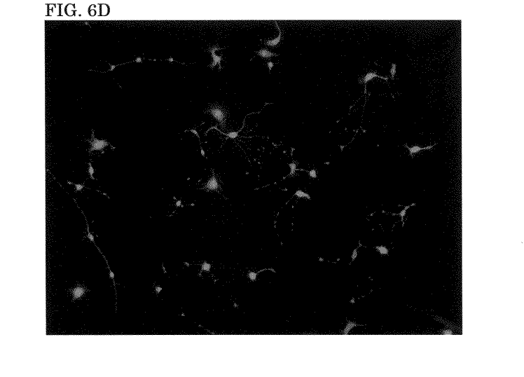

The hADSCs, in the control wells with no treatment, maintained their putative morphology as large, flat and spread cells on the culture surface, suggesting no obvious neurogenesis at this time point. See. FIG. 6A.

Early neurogenesis was observed three hours after switching to the neural differentiation medium with 20 μM uridine. See. FIG. 6B.

Notably, uridine at an experimental concentration of 0.5 mM demonstrated the strongest effect to enhance early neurogenesis. Moreover, such neuronal morpohological changes continued up to the 48-hour time course of the culture.

Similarly, uridine was found to promote neurogenesis on hNSCs. The hNSCs were cultured, passaged, seeded and exposed to uridine according to the described procedure in Example 3.

Uridine of 20 μM dramatically promoted neurogenesis, as the hNSCs appeared to have shrinking cellular bodies, projecting neurites, and developing dendrites. See FIG. 6C. The length of neurite outgrowth in the presence of uridine demonstrates good effects of neurogenesis.

Additionally, it was discovered that uridine is able to synergize with other brain nutrients to dramatically promote neurogenesis in a rapid manner. See. FIG. 6D, which shows the neuronal morphological changes after application of uridine together with 5 μM DHA, 0.1 μM N-octanoyl-D-threo-sphingosine, 25 μM cholesterol, 8.8 μM resveratrol, and 0.3 μM lutein in a purified or natural form. The neuronal morphological changes are shown as appearing more toward oligodendrocyte differentiation, suggesting a myelination function.

### Formulation Examples

Table 1 provides an example embodiment of a neurologic component that may be incorporated or added to the nutritional compositions described herein. This example provides the amount of each ingredient to be included per 100 kcal serving of nutritional composition.

TABLE 1
Nutrition profile of an example neurologic component

per 100 kcal

| Nutrient | Minimum | Maximum |
|---|---|---|
| PE (mg) | 3.7 | 37 |
| Sphingomyelin (mg) | 0.15 | 73 |
| CDP-Choline (mg) | 7 | 295 |
| Ceramide (mg) | 2.2 | 22 |
| Uridine (mg) | 0.15 | 37 |
| Gangliosides (mg) | 0.9 | 14.8 |

Table 2 provides an example embodiment of a nutritional composition according to the present disclosure and describes the amount of each ingredient to be included per 100 kcal serving.

TABLE 2
Nutrition profile of an example nutritional composition

per 100 kcal

| Nutrient | Minimum | Maximum |
|---|---|---|
| Protein (g) | 1.8 | 6.8 |
| Fat (g) | 1.3 | 7.2 |
| Carbohydrates (g) | 6 | 22 |
| Prebiotic (g) | 0.3 | 1.2 |
| DHA (g) | 4 | 22 |
| Beta glucan (mg) | 2.9 | 17 |
| PE (mg) | 3.7 | 37 |
| Sphingomyelin (mg) | 0.15 | 73 |
| CDP-Choline (mg) | 37 | 295 |
| Ceramide (mg) | 2.2 | 22 |
| Uridine (mg) | 0.7 | 37 |
| Probiotics (cfu) | 9.60 × 10⁵ | 3.80 × 10⁸ |
| Vitamin A (IU) | 134 | 921 |
| Vitamin D (IU) | 22 | 126 |
| Vitamin E (IU) | 0.8 | 5.4 |
| Vitamin K (mcg) | 2.9 | 18 |
| Thiamin (mcg) | 63 | 328 |
| Riboflavin (mcg) | 68 | 420 |
| Vitamin B6 (mcg) | 52 | 397 |
| Vitamin B12 (mcg) | 0.2 | 0.9 |
| Niacin (mcg) | 690 | 5881 |
| Folic acid (mcg) | 8 | 66 |
| Panthothenic acid (mcg) | 232 | 1211 |
| Biotin (mcg) | 1.4 | 5.5 |
| Vitamin C (mg) | 4.9 | 24 |
| Choline (mg) | 4.9 | 43 |
| Calcium (mg) | 68 | 297 |
| Phosphorus (mg) | 54 | 210 |
| Magnesium (mg) | 4.9 | 34 |
| Sodium (mg) | 24 | 88 |
| Potassium (mg) | 82 | 346 |
| Chloride (mg) | 53 | 237 |
| Iodine (mcg) | 8.9 | 79 |
| Iron (mg) | 0.7 | 2.8 |
| Zinc (mg) | 0.7 | 2.4 |
| Manganese (mcg) | 7.2 | 41 |
| Copper (mcg) | 16 | 331 |

All references cited in this specification, including without limitation, all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinence of the cited references.

Although embodiments of the disclosure have been described using specific terms, devices, and methods, such description is for illustrative purposes only. The words used are words of description rather than of limitation. It is to be understood that changes and variations may be made by those of ordinary skill in the art without departing from the spirit or the scope of the present disclosure, which is set forth in the following claims. In addition, it should be understood that aspects of the various embodiments may be interchanged in whole or in part. For example, while methods for the production of a commercially sterile liquid nutritional supplement made according to those methods have been exemplified, other uses are contemplated. Therefore, the spirit and scope of the appended claims should not be limited to the description of the versions contained therein.

1. A nutritional composition comprising:
(i) a carbohydrate source;
(ii) a protein source;
(iii) a fat source;
(iv) lactoferrin; and
(v) a neurologic component comprising lutein and a nutrient selected from the group consisting of PE, sphingomyelin, CDP-choline, ceramide, uridine, at least one ganglioside and combinations of one or more thereof, wherein the neurologic component promotes neurogenesis when provided to a target subject.

2. The nutritional composition of claim 1, wherein PE is present in an amount from about 3.7 mg/100 kcal to about 37 mg/100 kcal.

3. The nutritional composition of claim 1, wherein sphingomyelin is present in an amount from about 0.15 mg/100 kcal to about 73 mg/100 kcal.

4. The nutritional composition of claim 1, wherein CDP-choline is present in an amount from about 7 mg/100 kcal to about 295 mg/100 kcal.

5. The nutritional composition of claim 1, wherein ceramide is present in an amount from about 2.2 mg/100 kcal to about 22 mg/100 kcal.

6. The nutritional composition of claim 1, wherein uridine is present in an amount from about 0.15 mg/100 kcal to about 37 mg/100 kcal.

7. The nutritional composition of claim 1, wherein ceramide is selected from the group consisting of N-octanoyl-D-threo-sphingosine, N—(R,S) alpha-Hydroxydodecanoyl-D-erythro-sphingosine, and lactosylceramide.

8. The nutritional composition of claim 1, further comprising nutrients selected from the group consisting of a probiotic, a prebiotic, β-glucan, an iron source, and combinations of one or more thereof.

9. The nutritional composition of claim 1, further comprising nutrients selected from the group consisting of DHA, ARA, resveratrol, cholesterol, lutein, and combinations of at least one or more thereof.

10. The nutritional composition of claim 1, wherein the nutritional composition is an infant formula.

11. A nutritional composition, comprising per 100 kcal:
(i) between about 6 g and about 22 g of a carbohydrate source;
(ii) between about 1 g and about 7 g of a protein source;
(iii) between about 1.3 g and about 7.2 g of a fat source;
(iv) lactoferrin; and
(v) a neurologic component comprising:
(a) between about 3.7 mg and about 37 mg of PE;
(b) between about 0.15 mg and about 73 mg of sphingomyelin;
(c) between about 37 mg and about 295 mg CDP-choline;
(d) between about 2.2 mg and about 22 mg ceramide;
(e) between about 0.7 mg and about 37 mg of uridine;
(f) between about 0.9 mg and about 14.8 mg of at least one ganglioside; and
(g) lutein;
wherein the neurologic component promotes neurogenesis when provided to a target subject.

12. The nutritional composition of claim 11, further comprising per 100 kcal between about 9.60×10⁵ CFU and about 3.80×10⁸ CFU of probiotic.

13. The nutritional composition of claim 11, further comprising per 100 kcal between about 0.3 g and about 1.2 g of prebiotic.

14. The nutritional composition of claim 11, wherein the nutritional composition further comprises per 100 kcal between about 4 mg and about 50 mg of DHA.

15. The nutritional composition of claim 11, further comprising at least one nutrient selected from the group consisting of ARA, DHA, lutein, resveratrol, and cholesterol.

16. A method for promoting brain and nervous system health, comprising:
providing to a target subject, a nutritional composition comprising a carbohydrate source, a protein source, a fat source, lactoferrin, and a neurologic component, wherein the neurologic component comprises lutein and at least one nutrient selected from the group consisting of PE, sphingomyelin, CDP-choline, uridine, ceramide, at least one ganglioside, and combinations of one or more thereof, wherein the neurologic component promotes neurogenesis when provided to the target subject.

17. The method of claim 16, wherein the target subject is a pediatric subject.

18. The method of claim 16, wherein the nutritional composition is an infant formula.

19. The method of claim 16, wherein nutritional composition further comprises at least one nutrient selected from the group consisting of consisting of DHA, ARA, lutein, resveratrol, and cholesterol.

20. The method of claim 16, wherein the nutritional composition further comprises at least one nutrient selected from the group consisting of a probiotic, a prebiotic, β-glucan, and an iron source.
